(19) United States
(12) Patent Application Publication
(10) Pub. No.: US 2013/0172633 A1
(43) Pub. Date: Jul. 4, 2013

(54) Process For Producing Ethanol From Impure Methanol

(71) Applicants: Mark O. Scates, Houston, TX (US); James H. Zink, League City, TX (US)

(72) Inventors: Mark O. Scates, Houston, TX (US); James H. Zink, League City, TX (US)

(73) Assignee: CELANESE INTERNATIONAL CORPORATION, Irving, TX (US)

(21) Appl. No.: 13/592,829

(22) Filed: Aug. 23, 2012

Related U.S. Application Data
(60) Provisional application No. 61/581,161, filed on Dec. 29, 2011.

(51) Int. Cl. C07C 29/149

(52) U.S. Cl. 568/885

(57) ABSTRACT

In a first embodiment, the present invention relates to a process for producing ethanol. The process comprises the step of contacting a carbon monoxide feed and an impure methanol feed in a reactor under conditions effective to produce a crude acetic acid product. The impure methanol feed comprises more than 0.15 wt. % of impurities. The process further comprises the step of separating the crude acetic acid product to form an intermediate acetic acid product and at least one derivative stream. The intermediate acetic acid product may comprise acetic acid and at least one of the impurities from the impure methanol feed. The process further comprises the step of hydrogenating at least a portion of the intermediate acetic acid product to produce a crude ethanol product. The hydrogenation is preferably conducted over a catalyst.

### CROSS REFERENCE TO RELATED APPLICATION

This application claims priority to U.S. Prov. App. No. 61/581,161, filed on Dec. 29, 2011, the entire contents and disclosures of which are incorporated herein by reference.

### FIELD OF THE INVENTION

The present invention relates generally to alcohol production processes and, in particular, to ethanol production processes that integrate acetic acid production processes that utilize impure methanol feed streams to form a crude acetic acid product, which is then used to form the ethanol.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulosic materials, such as corn or sugar cane. Conventional methods for producing ethanol from petrochemical feed stocks, as well as from cellulosic materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulosic material, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. During the reduction of alkanoic acid, e.g., acetic acid, other compounds are often formed with ethanol or are formed in side reactions. These impurities may limit the production of ethanol and may require expensive and complex purification trains to separate the impurities from the ethanol.

Some processes for integrating acetic acid production and hydrogenation have been proposed in literature. Generally speaking, the conventional acetic acid production/separation processes yield a highly pure acetic acid product, which is then used as a feed to the hydrogenation process. The use of the highly pure acetic acid product limits the impurities that are formed in the subsequent hydrogenation.

For example, U.S. Pat. No. 7,884,253 discloses methods and apparatuses for selectively producing ethanol from syngas. The syngas is derived from cellulosic biomass (or other sources) and can be catalytically converted into methanol, which in turn can be catalytically converted into acetic acid or acetates. The ethanoic acid product may be removed from the reactor by withdrawing liquid reaction composition and separating the ethanoic acid product by one or more flash and/or fractional distillation stages from the other components of the liquid reaction composition such as iridium catalyst, ruthenium and/or osmium and/or indium promoter, methyl iodide, water and unconsumed reactants which may be recycled to the reactor to maintain their concentrations in the liquid reaction composition. As another example, EP2060553 discloses a process for the conversion of a carbonaceous feedstock to ethanol wherein the carbonaceous feedstock is first converted to ethanoic acid, which is then hydrogenated and converted into ethanol. Also, U.S. Pat. No. 4,497,967 discloses an integrated process for the preparation of ethanol from methanol, carbon monoxide and hydrogen feedstock. The process esterifies an acetic anhydride intermediate to form ethyl acetate and/or ethanol. In addition, U.S. Pat. No. 7,351,559 discloses a process for producing ethanol including a combination of biochemical and synthetic conversions results in high yield ethanol production with concurrent production of high value co-products. An acetic acid intermediate is produced from carbohydrates, such as corn, using enzymatic milling and fermentation steps, followed by conversion of the acetic acid into ethanol using esterification and hydrogenation reactions.

One conventional process for preparing acetic acid is methanol carbonylation, which reacts methanol and carbon monoxide to form acetic acid. Typically, methanol carbonylation processes employ highly pure methanol and/or carbon monoxide feed streams because impurities in the feeds may lead to: 1) build-up of impurities in the carbonylation process; and/or 2) undesired impurities in the resultant crude acetic acid product.

These highly pure methanol and/or carbon monoxide feed streams, however, are more expensive than less pure feeds. As such, the use of the highly pure feeds limits flexibility in raw material procurement.

In view of the conventional processes and literature, the need remains for improved ethanol production processes that are capable of effectively using acetic acid feed sources, which may be formed from less pure methanol and/or carbon monoxide feed sources.

### SUMMARY OF THE INVENTION

In one embodiment, the present invention relates to a process for producing ethanol. The process comprises the step of contacting a carbon monoxide feed and an impure methanol feed in a reactor under conditions effective to produce a crude acetic acid product. The impure methanol feed comprises more than 0.15 wt. % of impurities, e.g., organic impurities, chlorine-containing compounds, sulfur-containing compounds, and nitrogen containing compounds. In one embodiment, the impurities are selected from the group consisting of compounds having two or more carbon atoms. In one embodiment, organic compound(s) in the impure methanol that may lead to the formation of a compound other than acetic acid during carbonylation, e.g., methanol derivative(s), may not be considered impurities. The process further comprises the step of separating the crude acetic acid product to form an intermediate acetic acid product and optionally at least one derivative stream. The process further comprises the step of hydrogenating at least a portion of the intermediate acetic acid product to produce a crude ethanol product. The hydrogenation is preferably conducted over a catalyst.

The process further comprises the step of recovering an ethanol stream from the crude ethanol product. Preferably, the ethanol stream comprises less than 10 wt. % water.

The process further comprises the step of separating in a first column at least a portion of the crude ethanol product into a first distillate and a first residue. The first distillate comprises ethanol, water and ethyl acetate and the first residue comprises acetic acid. The process further comprises the step of separating in a second column at least a portion of the first distillate into a second distillate and a second residue. The second distillate comprises ethyl acetate and the second residue comprises ethanol and water. The process further comprises the step of separating in a third column at least a portion of the second residue into a third distillate and a third residue. The third distillate comprises ethanol and the third residue comprises water. The process further comprises the step of dehydrating at least a portion of the third distillate to form an anhydrous ethanol composition comprising less than 1 wt. % water, based on the total weight of the anhydrous ethanol composition.

In another embodiment, the process further comprises the step of separating at least a portion of the crude ethanol product in a first distillate column to yield a first residue and a first distillate. The first residue comprises acetic acid and a first distillate comprises ethanol, ethyl acetate, and water. The process further comprises the step of removing water from at least a portion of the first distillate to yield an ethanol mixture stream comprising less than 10 wt. % water. The process further comprises the step of separating a portion of the ethanol mixture stream in a second distillation column to yield a second residue and a second distillate. The second residue comprises ethanol and the second distillate comprises ethyl acetate.

In another embodiment, the process further comprises the step of separating a portion of the crude ethanol product in a first distillation column to yield a first distillate and a first residue. The first distillate comprises ethyl acetate and acetaldehyde and the first residue comprises ethanol, ethyl acetate, acetic acid and water. The process further comprises the step of separating a portion of the first residue in a second distillation column to yield a second residue and a second distillate. The second residue comprises acetic acid and water and the second distillate comprises ethanol and ethyl acetate. The process further comprises the step of separating a portion of the second distillate in a third distillation column to yield a third residue and a third distillate. The third residue comprises ethanol and the third distillate comprises ethyl acetate.

### BRIEF DESCRIPTION OF DRAWINGS

The invention is described in detail below with reference to the appended drawings, wherein like numerals designate similar parts.

FIG. 1 is a diagram of an acetic acid and ethanol integrated production process in accordance with one embodiment of the present invention.

FIG. 2 is a schematic diagram of an exemplary integrated carbonylation and hydrogenation process in accordance with one embodiment of the present invention.

FIG. 3 is a schematic diagram of a hydrogenation zone having at least four columns in accordance with one embodiment of the present invention.

FIG. 4 is a schematic diagram of a hydrogenation zone having two columns and an intervening water separation in accordance with one embodiment of the present invention.

FIG. 5 is a schematic diagram of a hydrogenation zone having at least two columns in accordance with one embodiment of the present invention.

### DETAILED DESCRIPTION OF THE INVENTION

### Introduction

In general, the present invention relates to hydrogenating acetic acid to form ethanol. During the hydrogenation, however, impurities are often formed via side reactions, and the resultant crude ethanol product comprises not only ethanol, but these impurities. These impurities are problematic because they may limit the production of ethanol and may require expensive and complex purification trains to separate the impurities from the ethanol.

One method to produce acetic acid is the carbonylation of methanol. In this reaction, carbon monoxide and methanol are reacted to form the acetic acid. Impurities in an acetic acid feed may result in impurities upon hydrogenation of the acetic acid feed. As such, to minimize the total impurities in the crude ethanol product, it is typically desirable to use an acetic acid feed that comprises only small amounts of impurities (if any). One method of forming a highly pure acetic acid feed is to use as reactants a highly pure methanol feed and a highly pure carbon monoxide feed. Although these highly pure methanol and/or carbon monoxide feed streams are useful and yield a desirable product, they are, however, more expensive than less pure feeds. As such, the use of the highly pure feeds limits flexibility in raw material procurement. In one embodiment, an impure methanol and/or carbon monoxide feed may be used to yield an intermediate acetic acid product. As such, the intermediate acetic acid product may contain the impurities in the methanol or carbon monoxide feeds.

The intermediate acetic acid product, in some embodiments, will have a higher impurity content as compared to that of conventional acetic acid feeds used in a hydrogenation reaction. Some impurities may not pass into the intermediate acetic acid product, but may build up in the carbonylation process. The removal of these impurities creates the need for additional separation units. For example, acetone may build up and/or carry through to the intermediate acetic acid product. Some impurities may react to form additional impurities in the crude acetic acid. For example, aldehydes in the methanol feed may hydrogenate to form ethanol, which may carbonylate to form propionic acid. Other examples of methanol impurities include aldehydes, unsaturates, and aromatics.

Regarding some specific methanol impurities, the methanol feed, in one embodiment, comprises more than 30 wppm acetone, e.g., more than 50 wppm, or more than 1000 wppm, as measured by International Methanol Producers & Consumers Association (IMPCA) 001-02. In one embodiment, the methanol feed comprises more than 0.1 wt. % water, e.g., more than 0.3 wt. % or more than 0.5 wt. %, as measured by ASTM D1209-05. In one embodiment, the methanol feed comprises more than 50 wppm ethanol, e.g., more than 100 wppm or more than 200 wppm, as measured by IMPCA 001-002. In one embodiment, the methanol feed comprises more than 0.5 wppm chlorine-containing compounds, e.g., more than 1 wppm or more than 10 wppm, as measured by IMPCA 002-98. In one embodiment, the methanol feed comprises more than 0.5 wppm sulphur-containing compounds, e.g., more than 1 wppm or more than 10 wppm, as measured by ASTM D3961-98 or ASTM D5453-09. In one embodiment, the methanol feed comprises more than 30 wppm carbonizable substances, e.g., more than 50 wppm or more than 100 wppm, as measured by ASTM E346-08. In one embodiment, the methanol feed comprises more than 30 wppm acetic acid, e.g., more than 50 wppm or more than 100 wppm, as measured by ASTM D1613-06. In one embodiment, the methanol feed comprises more than 0.1 wppm iron-containing compounds, e.g., more than 0.3 wppm or more than 0.5 wppm, as measured by ASTM D1613-06. In one embodiment, the methanol feed comprises more than 8 mg/1000 ml nonvolatile matter, e.g., more than 10 mg/1000 ml or more than 20 mg/1000 ml, as measured by ASTM D1353-09.

Surprisingly, the intermediate acetic acid product, including the organic impurities contained therein, may then be hydrogenated in accordance with the present invention to form a crude ethanol product that comprises a high concentration of ethanol and a low concentration of ethanol impurities. Less pure methanol and/or carbon monoxide feed streams can be employed to yield (via the intermediate acetic acid) suitable crude ethanol compositions. As a result, the cost of methanol feed can be reduced and procurement options improved. These less pure feeds may allow significant flexibility in raw material procurement, e.g., lower pricing and increased availability.

Accordingly, the present invention, in one embodiment, relates to a process for producing ethanol. The process comprises the step of contacting a carbon monoxide feed and an impure methanol feed in a reactor under conditions effective to produce a crude acetic acid product. In one embodiment, the impure methanol feed is preferably a lower purity methanol feed and comprises more than 0.15 wt. % impurities, e.g., more than 0.25 wt. % or more than 0.5 wt. %. In terms of ranges, the methanol feed may comprise from 0.15 wt. % to 5 wt. % impurities, e.g., from 0.25 wt. % to 5 wt. % or from 0.5 wt. % to 3 wt. %. In one embodiment, the organic impurities may be selected from the group consisting of organic impurities, chlorine-containing compounds, sulfur-containing compounds, and nitrogen-containing compounds. In one embodiment, the organic impurities are selected from the group consisting of hydrocarbons having two or more carbon atoms. In one embodiment, an (organic) impurity in the impure methanol may lead to the formation of a compound other than acetic acid during carbonylation. For example, the impurity may be a methanol derivative. Methanol derivative(s), e.g., methyl acetate, dimethyl ether, and methyl formate, in some embodiments, may not be considered impurities for the purposes of the present invention. Of course, methanol must be present in order to conduct the desired reaction. In terms of ranges, the impure methanol feed comprises from 90 wt. % to 99.99 wt. % methanol, e.g., from 95 wt. % to 99.9 wt. % or from 98 wt. % to 99.85 wt. %.

In one embodiment, the carbon monoxide feed may also be a lower purity carbon monoxide feed (as compared to a conventional carbon monoxide feed stream), e.g., a carbon monoxide feed comprising less than 99.5 wt. % carbon monoxide, e.g., less than 95 wt. %, less than 90 wt. % or less than 60 wt. %. Of course, some carbon monoxide must be present in order to conduct the desired reaction. As a lower limit, the carbon monoxide feed may comprise more than 10 wt. % carbon monoxide, e.g., more than 20 wt. %, more than 50 wt. % or more than 75 wt. %. In terms of ranges, the carbon monoxide feed may comprise from 10 wt. % to 99.5 wt. % carbon monoxide, e.g., from 50 wt. % to 95 wt. %, from 75 wt. % to 90 wt. %, or from 60 wt. % to 70 wt. %. The lower purity carbon monoxide feeds of the present invention comprise lower amounts of carbon monoxide and/or higher amounts of impurities than conventional carbon monoxide feeds. In one embodiment, however, the carbon monoxide feed is a conventional carbon monoxide feed, e.g., a carbon monoxide feed comprising from 10 wt. % to 99.99 wt. % carbon monoxide, e.g., from 20 wt. % to 99 wt. %.

The process further comprises the step of separating the crude acetic acid product to form an intermediate acetic acid product and optionally at least one derivative stream. The separation of the crude acetic acid may be performed in one or more, e.g., two or more, separation units. Exemplary separation units include a light ends column, a phase separator, and/or a drying column. The intermediate acetic acid product may result from any one of the separation units or from any combination of the separation unit(s). As an example, the intermediate acetic acid product may be a sidedraw from a light ends column. As another example, the intermediate acetic acid product may be a purified vapor stream from a phase separator.

In one embodiment, the intermediate acetic acid product is a low purity acetic acid product that comprises acetic acid and at least one of: 1) the impurities from the impure methanol stream; and 2) by-products formed from the methanol impurities during the carbonylation reaction. In one embodiment, the impurities in the intermediate acetic acid product comprise aldol condensation products that may be formed as by-products of the carbonylation. For example, the impurities may comprise crotonaldehyde, butaldehyde, 3-penten-2-one, 2-butanone, isobutyl aldehyde, butyl aldehyde, and/or 4-methyl-3-penten-2-one. Some of these impurities may be caused by a methanol feed that comprises acetone. In one embodiment, nitrogen-containing compounds, e.g., trimethyl amine, present in the methanol feed may pass through to the intermediate acetic acid product. In general, the intermediate acetic acid product comprises more than 0.1 wt. % impurities, e.g., more than 0.5 wt. % or more than 1 wt. %. In terms of ranges, the intermediate acetic acid product may comprise from 0.1 wt. % to 10 wt. % organic impurities, e.g., from 0.5 wt. % to 10 wt. % or from 1 wt. % to 5 wt. %. Of course, some acetic acid must be present in order to conduct the desired reaction. In terms of ranges, the intermediate acetic acid product comprises from 90 wt. % to 99.9 wt. % acetic acid, e.g., from 90 wt. % to 99.5 wt. % or from 95 wt. % to 99.5 wt. %.

The process further comprises the step of hydrogenating at least a portion of the intermediate acetic acid product to produce a crude ethanol product. In some embodiments, some of the impurities may also be hydrogenated to ethanol. The hydrogenation is preferably conducted over a catalyst. In some embodiments, some of the impurities are carried over to the crude ethanol product.

In one embodiment, the at least one derivative stream comprises residual carbon monoxide, e.g., unreacted carbon monoxide from the carbonylation reaction. Preferably, at least a portion of the residual carbon monoxide may be further processed, e.g., reacted, to form additional acetic acid. An example of such a reaction is disclosed in U.S. Pub. No. 2012/0078012, which is hereby incorporated by reference. In one embodiment, the additional acetic acid, thus produced, is hydrogenated over a catalyst and under conditions effective to produce additional ethanol.

The use of a lower purity methanol feed yields a crude acetic acid product, and subsequently an intermediate acetic acid product, that is less pure than a conventional acetic acid product that is fed to a hydrogenation reaction. The increased impurity level in 1) the methanol feed; and/or 2) the intermediate acetic acid product would be expected to be detrimental to ethanol production. However, without being bound by theory, feeding such an intermediate acetic acid product containing organic impurities from the impure ethanol to a hydrogenation reactor does not substantially affect the conversion of acetic acid to ethanol.

In one embodiment, the intermediate acetic acid product further comprises water in amounts of up to 25 wt. %, e.g., up to 20 wt. % water, or up to 10 wt. % water. In some embodiments, the water content may be very low and may be less than 1500 wppm water, e.g., less than 1000 wppm or less than 500 wppm. In other embodiment there may be relatively more water present and in terms of ranges the intermediate acetic acid product may comprise from 0.15 wt. % to 25 wt. % water, e.g., from 0.2 wt. % to 20 wt. %, from 0.5 to 15 wt. %, or from 4 wt. % to 10. wt. %. In one embodiment, the acetic acid feed stream that is provided to the ethanol production process comprises water in an amount of at least 1500 wppm, e.g., at least 2500 wppm, at least 5000 wppm, or at least 1 wt. %. In some embodiments, the intermediate acetic acid product may also comprise other carboxylic acids, anhydrides, aldehyde and/or ketones. In particular, the intermediate acetic acid product may comprise methyl acetate and/or propanoic acid. In one embodiment, the intermediate acetic acid product comprises from 0.01 to 10 wt. % methyl acetate, e.g., from 0.1 wt % to 10 wt % or from 1 wt % to 5 wt %. These other compounds may also be hydrogenated in the processes of the present invention.

### Carbonylation

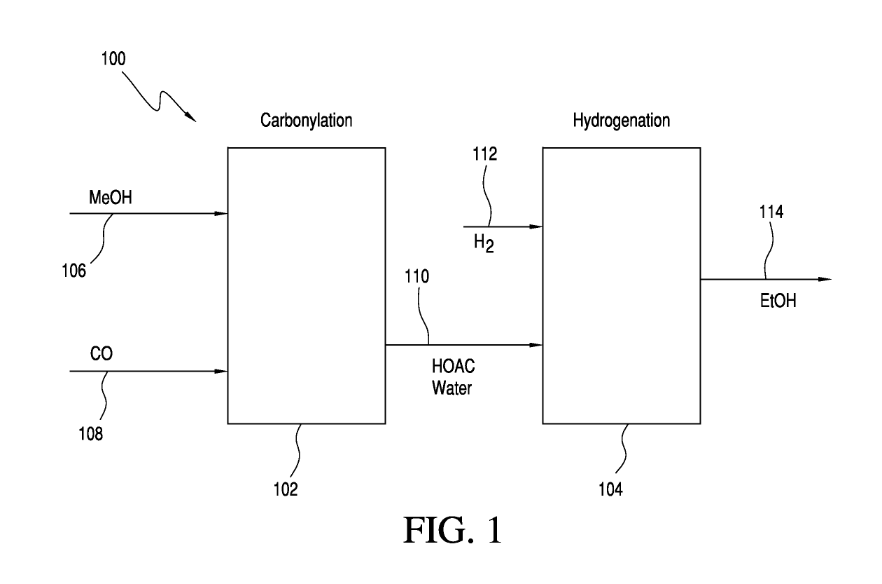

FIG. 1 is a diagram of an integrated process 100 in accordance with the present invention. Process 100 comprises carbonylation system 102 and hydrogenation system 104. Carbonylation system 102 receives impure methanol feed 106 and/or impure carbon monoxide feed 108. In one embodiment, the impure methanol feed and/or the impure carbon monoxide feeds are low purity feeds, as discussed above. The methanol and the carbon monoxide are reacted in carbonylation zone 102 to form intermediate acetic acid product 110 comprising acetic acid and impurities. A flasher may be used to remove residual catalyst from intermediate acetic acid product 110. Carbonylation system 102, in some embodiments, further comprises a purification train comprising one or more distillation columns (not explicitly shown in FIG. 1) to separate crude product into intermediate acetic acid product 110. Generally, the impurities from impure methanol feed 106 may be passed along into the hydrogenation system 104.

Intermediate acetic acid product 110 is fed, preferably directly fed, to hydrogenation system 104. Hydrogenation system 104 also receives hydrogen feed 112. In hydrogenation system 104, the acetic acid in intermediate acetic acid product 110 is hydrogenated to form a crude ethanol product comprising ethanol and other compounds such as water, ethyl acetate, and unreacted acetic acid. Hydrogenation system 104 further comprises one or more separation units, e.g. distillation columns, (not explicitly shown in FIG. 1) for recovering ethanol from the crude ethanol product. These distillation columns may also remove the organic impurities from impure methanol feed 104. Once separated, a purified ethanol product stream exits hydrogenation system 104 as shown by stream 114.

The process of the present invention may be used with any hydrogenation process for producing ethanol. The materials, catalysts, reaction conditions, and separation processes that may be used in the hydrogenation of acetic acid are described further below.

The raw materials, acetic acid and hydrogen, used in connection with the process of this invention may be derived from any suitable source including natural gas, petroleum, coal, biomass, and so forth. For purposes of the present invention, acetic acid may be produced using an impure methanol feed via methanol carbonylation as described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

Biomass-derived syngas has a detectable ¹⁴C isotope content as compared to fossil fuels such as coal or natural gas. An equilibrium forms in the Earth's atmosphere between constant new formation and constant degradation, and so the proportion of the ¹⁴C nuclei in the carbon in the atmosphere on Earth is constant over long periods. The same distribution ratio n¹⁴C:n¹²C ratio is established in living organisms as is present in the surrounding atmosphere, which stops at death and ¹⁴C decomposes at a half life of about 6000 years. Methanol, acetic acid and/or ethanol formed from biomass-derived syngas would be expected to have a ¹⁴C content that is substantially similar to living organisms. For example, the ¹²C ratio of the methanol, acetic acid and/or ethanol may be from one half to about 1 of the ¹⁴C:¹²C ratio for living organisms. In other embodiments, the syngas, methanol, acetic acid and/or ethanol described herein are derived wholly from fossil fuels, i.e. carbon sources produced over 60,000 years ago, may have no detectable ¹⁴C content.

In another embodiment, in addition to the acetic acid formed via methanol carbonylation, some additional acetic acid may be formed from the fermentation of biomass and may be used in the hydrogenation step. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. No. 6,509,180, and U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. Another biomass source is black liquor, which is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into syngas, and U.S. Pat. No. 6,685,754, which discloses a method for the production of a hydrogen-containing gas composition, such as syngas including hydrogen and carbon monoxide, are incorporated herein by reference in their entireties.

Acetic acid fed to the hydrogenation reaction may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

Although carbonylation may be a preferred acetic acid production method, other suitable methods may be employed. In a preferred embodiment that employs carbonylation, the carbonylation system preferably comprises a reaction zone, which includes a reactor, a flasher and optionally a reactor recovery unit. In one embodiment, carbon monoxide is reacted with methanol in a suitable reactor, e.g., a continuous stirred tank reactor (“CSTR”) or a bubble column reactor. Preferably, the carbonylation process is a low water, catalyzed, e.g., rhodium-catalyzed, carbonylation of methanol to acetic acid, as exemplified in U.S. Pat. No. 5,001,259, which is hereby incorporated by reference.

The carbonylation reaction may be conducted in a homogeneous catalytic reaction system comprising a reaction solvent, methanol and/or reactive derivatives thereof, a Group VIII catalyst, at least a finite concentration of water, and optionally an iodide salt. Preferably, methanol is obtained from an impure methanol feed that is not purified prior to carbonylation.

Suitable catalysts include Group VIII catalysts, e.g., rhodium and/or iridium catalysts. When a rhodium catalyst is utilized, the rhodium catalyst may be added in any suitable form such that the active rhodium catalyst is a carbonyl iodide complex. Exemplary rhodium catalysts are described in Michael Gauβ, et al., Applied Homogeneous Catalysis with Organometallic Compounds: A Comprehensive Handbook in Two Volume, Chapter 2.1, p. 27-200, (1^{st} ed., 1996). Iodide salts optionally maintained in the reaction mixtures of the processes described herein may be in the form of a soluble salt of an alkali metal or alkaline earth metal or a quaternary ammonium or phosphonium salt. In certain embodiments, a catalyst co-promoter comprising lithium iodide, lithium acetate, or mixtures thereof may be employed. The salt co-promoter may be added as a non-iodide salt that will generate an iodide salt. The iodide catalyst stabilizer may be introduced directly into the reaction system. Alternatively, the iodide salt may be generated in-situ since under the operating conditions of the reaction system, a wide range of non-iodide salt precursors will react with methyl iodide or hydroiodic acid in the reaction medium to generate the corresponding co-promoter iodide salt stabilizer. For additional detail regarding rhodium catalysis and iodide salt generation, see U.S. Pat. Nos. 5,001,259; 5,026,908; and 5,144,068, which are hereby incorporated by reference.

When an iridium catalyst is utilized, the iridium catalyst may comprise any iridium-containing compound which is soluble in the liquid reaction composition. The iridium catalyst may be added to the liquid reaction composition for the carbonylation reaction in any suitable form which dissolves in the liquid reaction composition or is convertible to a soluble form. Examples of suitable iridium-containing compounds which may be added to the liquid reaction composition include: IrCl₃, IrI₃, IrBr₃, [Ir(CO)₂I]₂, [Ir(CO)₂Cl]₂, [Ir(CO)₂Br]₂, [Ir(CO)₂I₂]H⁺, [Ir(CO)₂Br₂]⁻H⁺, [Ir(CO)₂I₄]⁻H⁺, [Ir(CH₃)I₃(CO₂]⁻H⁺, Ir₄(CO)₁₂, IrCl₃.3H₂O, IrBr₃.3H₂O, iridium metal, Ir₂O₃, Ir(acac)(CO)₂, Ir(acac)₃, iridium acetate, [Ir₃O(OAc)₆(H₂O)₃][OAc], and hexachloroiridic acid [H₂IrCl₆]. Chloride-free complexes of iridium such as acetates, oxalates and acetoacetates are usually employed as starting materials. The iridium catalyst concentration in the liquid reaction composition may be in the range of 100 to 6000 ppm. The carbonylation of methanol utilizing iridium catalyst is well known and is generally described in U.S. Pat. Nos. 5,942,460; 5,932,764; 5,883,295; 5,877,348; 5,877,347; and 5,696,284, which are hereby incorporated by reference.

A halogen co-catalyst/promoter is generally used in combination with the Group VIII metal catalyst component. Methyl iodide is a preferred halogen promoter. Preferably, the concentration of halogen promoter in the reaction medium ranges from 1 wt. % to 50 wt. %, and preferably from 2 wt. % to 30 wt. %.

The halogen promoter may be combined with the salt stabilizer/co-promoter compound. Particularly preferred are iodide or acetate salts, e.g., lithium iodide or lithium acetate.

Other promoters and co-promoters may be used as part of the catalytic system of the present invention as described in U.S. Pat. No. 5,877,348, which is hereby incorporated by reference. Suitable promoters are selected from ruthenium, osmium, tungsten, rhenium, zinc, cadmium, indium, gallium, mercury, nickel, platinum, vanadium, titanium, copper, aluminum, tin, antimony, and are more preferably selected from ruthenium and osmium. Specific co-promoters are described in U.S. Pat. No. 6,627,770, which is incorporated herein by reference.

A promoter may be present in an effective amount up to the limit of its solubility in the liquid reaction composition and/or any liquid process streams recycled to the carbonylation reactor from the acetic acid recovery stage. When used, the promoter is suitably present in the liquid reaction composition at a molar ratio of promoter to metal catalyst of 0.5:1 to 15:1, preferably 2:1 to 10:1, more preferably 2:1 to 7.5:1. A suitable promoter concentration is 400 to 5000 ppm.

In one embodiment, the temperature of the carbonylation reaction in the reactor is preferably from 150° C. to 250° C., e.g., from 150° C. to 225° C., or from 150° C. to 200° C. The pressure of the carbonylation reaction is preferably from 1 to 20 MPa, preferably 1 to 10 MPa, most preferably 1.5 to 5 MPa. Acetic acid is typically manufactured in a liquid phase reaction at a temperature from about 150° C. to about 200° C. and a total pressure from about 2 to about 5 MPa.

In one embodiment, reaction mixture comprises a reaction solvent or mixture of solvents. The solvent is preferably compatible with the catalyst system and may include pure alcohols, mixtures of an alcohol feedstock, and/or the desired carboxylic acid and/or esters of these two compounds. In one embodiment, the solvent and liquid reaction medium for the (low water) carbonylation process is preferably acetic acid.

Water may be formed in situ in the reaction medium, for example, by the esterification reaction between methanol reactant and acetic acid product. In some embodiments, water is introduced to reactor together with or separately from other components of the reaction medium. Water may be separated from the other components of reaction product withdrawn from reactor and may be recycled in controlled amounts to maintain the required concentration of water in the reaction medium. Preferably, the concentration of water maintained in the reaction medium ranges from 0.1 wt. % to 16 wt. %, e.g., from 1 wt. % to 14 wt. %, or from 1 wt. % to 3 wt. % of the total weight of the reaction product.

The desired reaction rates are obtained even at low water concentrations by maintaining in the reaction medium an ester of the desired carboxylic acid and an alcohol, desirably the alcohol used in the carbonylation, and an additional iodide ion that is over and above the iodide ion that is present as hydrogen iodide. An example of a preferred ester is methyl acetate. The additional iodide ion is desirably an iodide salt, with lithium iodide (LiI) being preferred. It has been found, as described in U.S. Pat. No. 5,001,259, that under low water concentrations, methyl acetate and lithium iodide act as rate promoters only when relatively high concentrations of each of these components are present and that the promotion is higher when both of these components are present simultaneously. The absolute concentration of iodide ion content is not a limitation on the usefulness of the present invention.

In low water carbonylation, the additional iodide over and above the organic iodide promoter may be present in the catalyst solution in amounts ranging from 2 wt. % to 20 wt. %, e.g., from 2 wt. % to 15 wt. %, or from 3 wt. % to 10 wt. %; the methyl acetate may be present in amounts ranging from 0.5 wt % to 30 wt. %, e.g., from 1 wt. % to 25 wt. %, or from 2 wt. % to 20 wt. %; and the lithium iodide may be present in amounts ranging from 5 wt. % to 20 wt %, e.g., from 5 wt. % to 15 wt. %, or from 5 wt. % to 10 wt. %. The catalyst may be present in the catalyst solution in amounts ranging from 200 wppm to 2000 wppm, e.g., from 200 wppm to 1500 wppm, or from 500 wppm to 1500 wppm.

### Hydrogenation of Acetic Acid

The carbonylation system may be integrated with an acetic acid hydrogenation process to produce ethanol with the following exemplary hydrogenation reaction conditions and catalysts.

The acetic acid, along with water and organic impurities from the methanol feed, may be vaporized at the reaction temperature, following which the vaporized acetic acid can be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol according to one embodiment of the invention may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 18:1 to 2:1.

Contact or residence time can also vary widely, depending upon such variables as amount of acetic acid, catalyst, reactor, temperature and pressure. Typical contact times range from a fraction of a second to more than several hours when a catalyst system other than a fixed bed is used, with preferred contact times, at least for vapor phase reactions, from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Exemplary catalysts are further described in U.S. Pat. Nos. 7,608,744 and 7,863,489, and U.S. Pub. Nos. 2010/0121114 and 2010/0197985, the entireties of which are incorporated herein by reference. In another embodiment, the catalyst comprises a Co/Mo/S catalyst of the type described in U.S. Pub. No. 2009/0069609, the entirety of which is incorporated herein by reference. In some embodiments the catalyst may be a bulk catalyst.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %.

Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron.

The catalyst may also comprise a third metal selected from any of the metals listed above in connection with the first or second metal, so long as the third metal is different from both the first and second metals. In preferred embodiments, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 20 wt. %, e.g., from 0.1 to 10 wt. %, or from 0.1 to 7.5 wt. %. In one embodiment, the catalyst may comprise platinum, tin and cobalt.

In addition to one or more metals, in some embodiments of the present invention, the catalysts further comprise a support or a modified support. As used herein, the term “modified support” refers to a support that includes a support material and a support modifier, which adjusts the acidity of the support material. The total weight of the support or modified support, based on the total weight of the catalyst, preferably is from 75 to 99.9 wt. %, e.g., from 78 to 99 wt. %, or from 80 to 97.5 wt. %. In preferred embodiments that utilize a modified support, the support modifier is present in an amount from 0.1 to 50 wt. %, e.g., from 0.2 to 25 wt. %, from 1 to 20 wt. %, or from 3 to 15 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica gel, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

The support may be a modified support and the support modifier is present in an amount from 0.1 to 50 wt. %, e.g., from 0.2 to 25 wt. %, from 1 to 20 wt. %, or from 3 to 15 wt. %, based on the total weight of the catalyst. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃. Preferred support modifiers include oxides of tungsten, molybdenum, and vanadium.

In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. The basic support modifier may be selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

Catalysts on a modified support may include one or more metals from the group of platinum, palladium, cobalt, tin, or rhenium on a silica support modified by one or more modifiers from the group of calcium metasilicate, oxides of tungsten, molybdenum, and vanadium.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197985 referred to above, the entireties of which are incorporated herein by reference.

After the washing, drying and calcining of the catalyst is completed, the catalyst may be reduced in order to activate the catalyst. Reduction is carried out in the presence of a reducing gas, preferably hydrogen. The reducing gas is continuously passed over the catalyst at an initial ambient temperature that is increased up to about 400° C. In one embodiment, the reduction is preferably carried out after the catalyst has been loaded into the reaction vessel where the hydrogenation will be carried out.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a percentage based on acetic acid in the feed. The conversion may be at least 40%, e.g., at least 50%, at least 60%, at least 70% or at least 80%. Although catalysts that have high conversions are desirable, such as at least 80% or at least 90%, in some embodiments a low conversion may be acceptable at high selectivity for ethanol.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethanol is at least 60%, e.g., at least 70%, or at least 80%. Preferred embodiments of the hydrogenation process also have low selectivity to undesirable products, such as methane, ethane, and carbon dioxide. The selectivity to these undesirable products preferably is less than 4%, e.g., less than 2% or less than 1%.

The term “productivity,” as used herein, refers to the grams of a specified product, e.g., ethanol, formed during the hydrogenation based on the kilograms of catalyst used per hour. The productivity may range from 100 to 3,000 grams of ethanol per kilogram of catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol stream produced by the hydrogenation process, before any subsequent processing, such as purification and separation, will typically comprise unreacted acetic acid, ethanol and water. Exemplary component ranges for the crude ethanol product are provided in Table 1, excluding hydrogen. The “others” identified in Table 1 may include, for example, esters, ethers, aldehydes, ketones, alkanes, and carbon dioxide.

TABLE 1
CRUDE ETHANOL PRODUCT

| | Conc. | Conc. | | |
|---|---|---|---|---|
| Component | (wt. %) | (wt. %) | Conc. (wt. %) | Conc. (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 18 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product comprises acetic acid in an amount less than 20 wt. %, e.g., less than 15 wt. %, less than 10 wt. % or less than 5 wt. %. In terms of ranges, the acetic acid concentration of Table 1 may range from 0.1 wt. % to 20 wt. %, e.g., 0.2 wt. % to 15 wt. %, from 0.5 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is preferably greater than 75%, e.g., greater than 85% or greater than 90%.

### Integration of Carbonylation and Hydrogenation

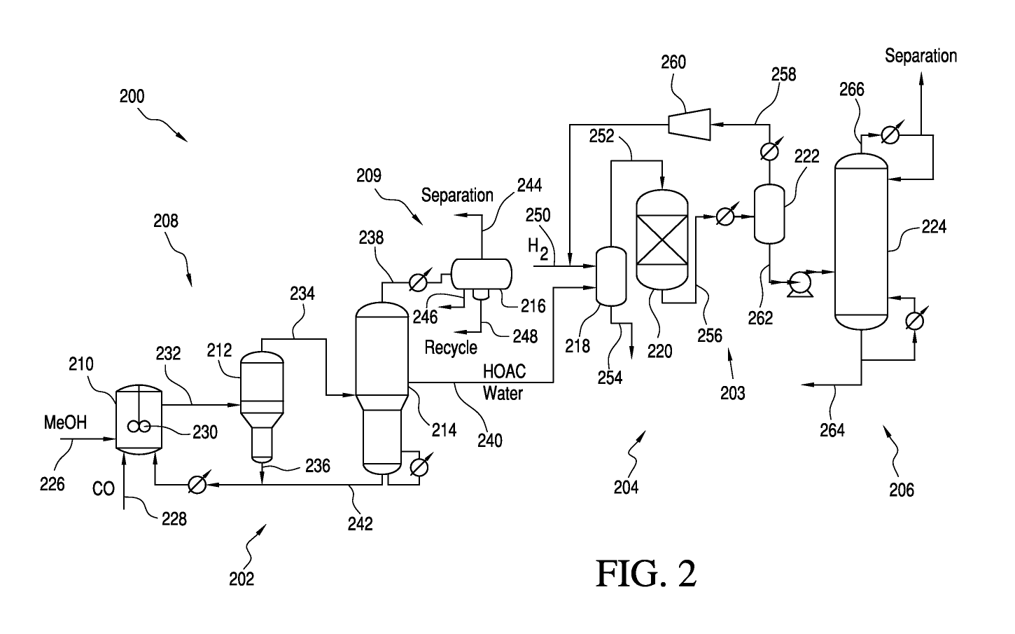

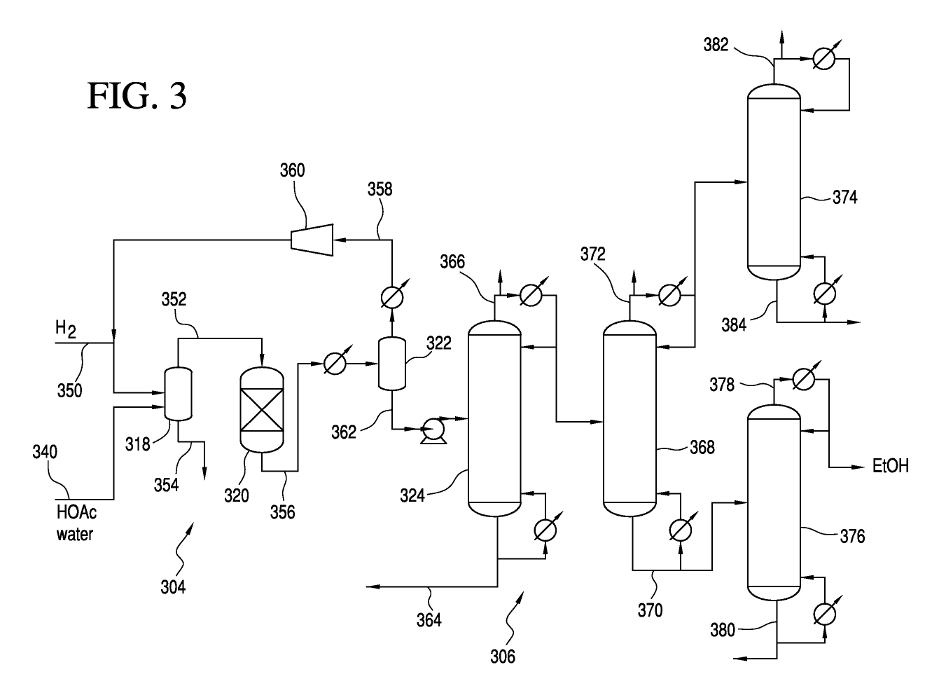

FIG. 2 shows exemplary integrated carbonylation and hydrogenation process 200, which comprises carbonylation system 202 and hydrogenation system 204, which comprises hydrogenation reaction zone 203 and hydrogenation separation zone 206. Carbonylation system 202 comprises reaction zone 208 and carbonylation zone 209. Reaction zone 208 comprises carbonylation reactor 210 and flasher 212, and carbonylation separation zone 209 comprises at least one distillation column, e.g., a light ends column or a drying column, 214, and phase separator, e.g., decanter, 216. Hydrogenation reaction zone 203 comprising vaporizer 218 and hydrogenation reactor 220. Hydrogenation separation zone 206 comprises flasher 222 and column 224, also referred to as an “acid separation column.” FIGS. 3-5 show exemplary hydrogenation systems having multiple columns as described herein.

Returning to FIG. 2, in carbonylation system 202, impure methanol feed stream 226 comprising methanol and one or more organic impurities and carbon monoxide feed stream 228 are fed to a lower portion of carbonylation reactor 210. At least some of the methanol may be converted to, and hence present as, methyl acetate in the liquid reaction composition by reacting with acetic acid product or solvent. The concentration in the liquid reaction composition of methyl acetate is suitably in the range from 0.5 wt. % to 70 wt. %, e.g., from 0.5 wt. % to 50 wt. %, from 1 wt. % to 35 wt. %, or from 1 wt. % to 20 wt. %.

Reactor 210 is preferably either a stirred vessel, e.g., CSTR, or bubble-column type vessel, with agitator 230 or without an agitator, within which the reaction medium is maintained, preferably automatically, at a predetermined level. This predetermined level may remain substantially constant during normal operation. Into reactor 210, impure methanol, carbon monoxide, and sufficient water may be continuously introduced as needed to maintain at least a finite concentration of water in the reaction medium. In one embodiment, carbon monoxide, e.g., in the gaseous state, is continuously introduced into reactor 210, desirably below agitator 230, which is used to stir the contents. The temperature of reactor 210 may be controlled, as indicated above. Carbon monoxide feed 228 is introduced at a rate sufficient to maintain the desired total reactor pressure.

The gaseous carbon monoxide feed is preferably thoroughly dispersed through the reaction medium by agitator 230. A gaseous purge is desirably vented via an off-gas line (not shown) from reactor 210 to prevent buildup of gaseous by-products, such as methane, carbon dioxide, and hydrogen, and to maintain a carbon monoxide partial pressure at a given total reactor pressure.

The crude acetic acid product is drawn off from the reactor 210 at a rate sufficient to maintain a constant level therein and is provided to flasher 212 via stream 232. The crude acetic acid product has the compositions discussed above.

In flasher 212, the crude acetic acid product is separated in a flash separation step to obtain a volatile (“vapor”) overhead stream 234 comprising acetic acid and a less volatile stream 236 comprising a catalyst-containing solution. Impurities from the methanol feed may be passed into overhead stream 234. In one embodiment, overhead stream 234 may be considered an intermediate acetic acid product stream, as discussed above. The catalyst-containing solution comprises acetic acid containing the rhodium and the iodide salt along with lesser quantities of methyl acetate, methyl iodide, and water. The less volatile stream 236 preferably is recycled to reactor 210. Vapor overhead stream 234 also comprises methyl iodide, methyl acetate, water, and permanganate reducing compounds (“PRCs”).

Overhead stream 234 from flasher 212 is directed to separation zone 209. Separation zone 209 comprises light ends column 214 and decanter 216. Separation zone 209 may also comprise additional units, e.g., a drying column, one or more columns for removing PRCs, heavy ends columns, extractors, etc.

In light ends column 214, intermediate acetic acid product stream 234 yields a low-boiling overhead vapor stream 238, a purified acetic acid stream, which preferably is removed via a sidestream 240, and a high boiling residue stream 242. In one embodiment, the purified acetic acid product that is removed via sidestream 240 preferably is conveyed, e.g., directly, without removing substantially any water therefrom, to hydrogenation system 204, e.g., reaction zone 203 of hydrogenation system 204. In some embodiments, there may be a production efficiency increase by using an acetic acid stream having a higher water content than glacial acetic acid, which beneficially reduces or eliminates the need for water removal downstream from light ends column 214 in carbonylation system 202.

In one embodiment, column 214 may comprise trays having different concentrations of water. In these cases, the composition of a withdrawn sidedraw may vary throughout the column. As such, the withdrawal tray may be selected based on the amount of water that is desired, e.g., more than 0.5 wt %. In another embodiment, the configuration of the column may be varied to achieve a desired amount or concentration of water in a sidedraw. Thus, an acetic acid feed may be produced, e.g., withdrawn from a column, based on a desired water content. Accordingly, in one embodiment, the invention is to a process for producing ethanol comprising the step of withdrawing a purified acetic acid sidedraw from a light ends column in a carbonylation process, wherein a location from which the sidedraw is withdrawn is based on a water content of the sidedraw. The water content of the sidedraw may be from 0.15 wt. % to 25 wt. % water. The process further comprises the steps of hydrogenating acetic acid of the purified acetic acid stream in the presence of a catalyst under conditions effective to form a crude ethanol product comprising ethanol and water; and recovering ethanol from the crude ethanol product.

In another embodiment, separation zone 209 comprises a second column, such as a drying column (not shown). A portion of the crude acetic acid stream 240 may be directed to the second column to separate some of the water from sidedraw 240 as well as other components such as esters and halogens. In these cases, the drying column may yield an acetic acid residue comprising acetic acid and less than 1500 wppm water. Depending on how the drying column is operated, water concentration may be increased to within the range from 0.15 wt. % to 25 wt. %. The acetic acid residue exiting the second column may be fed to hydrogenation system 204 in accordance with the present invention.

The purified acetic acid stream, in some embodiments, comprises methyl acetate, e.g., in an amount ranging from 0.01 wt. % to 10 wt. % or from 0.1 wt. % to 5 wt. %. This methyl acetate, in preferred embodiments, may be reduced to form methanol and/or ethanol. In addition to acetic acid, water, and methyl acetate, the purified acetic acid stream may comprise halogens, e.g., methyl iodide, which may be removed from the purified acetic acid stream.

Returning to column 214, low-boiling overhead vapor stream 238 is preferably condensed and directed to an overhead phase separation unit, as shown by overhead receiver decanter 216. Conditions are desirably maintained in the process such that low-boiling overhead vapor stream 238, once in decanter 216, will separate into a light phase and a heavy phase. Generally, low-boiling overhead vapor stream 238 is cooled to a temperature sufficient to condense and separate the condensable methyl iodide, methyl acetate, acetaldehyde and other carbonyl components, and water into two phases. A gaseous portion of stream 238 may include carbon monoxide, and other noncondensable gases such as methyl iodide, carbon dioxide, hydrogen, and the like and is vented from the decanter 216 via stream 244.

Condensed light phase 246 from decanter 216 preferably comprises water, acetic acid, and permanganate reducing compounds (“PRCs”), as well as quantities of methyl iodide and methyl acetate. Condensed heavy phase 248 from decanter 216 will generally comprise methyl iodide, methyl acetate, and PRCs. The condensed heavy liquid phase 248, in some embodiments, may be recirculated, either directly or indirectly, to reactor 210. For example, a portion of condensed heavy liquid phase 248 can be recycled to reactor 210, with a slip stream (not shown), generally a small amount, e.g., from 5 to 40 vol. %, or from 5 to 20 vol. %, of the heavy liquid phase being directed to a PRC removal system. This slip stream of heavy liquid phase 248 may be treated individually or may be combined with condensed light liquid phase 246 for further distillation and extraction of carbonyl impurities in accordance with one embodiment of the present invention.

Acetic acid sidedraw 240 from distillation column 214 of carbonylation process 202 is preferably directed to hydrogenation system 204. In one embodiment, the purified acetic acid stream may be sidestream 240 from a light ends column 214.

In hydrogenation system 204, hydrogen feed line 250 and sidedraw 240 comprising acetic acid and water is fed to vaporizer 218. Vapor feed stream 252 is withdrawn and fed to hydrogenation reactor 220. In one embodiment, lines 250 and 240 may be combined and jointly fed to the vaporizer 218. The temperature of vapor feed stream 252 is preferably from 100° C. to 350° C., e.g., from 120° C. to 310° C. or from 150° C. to 300° C. Vapor feed stream 252 comprises from 0.15 wt. % to 25 wt. % water. Any feed that is not vaporized is removed from vaporizer 218 via stream 254, as shown in FIG. 2, and may be recycled thereto or discarded. In addition, although FIG. 2 shows line 252 being directed to the top of reactor 220, line 252 may be directed to the side, upper portion, or bottom of reactor 220. Further modifications and additional components to reaction zone 204 are described below.

Reactor 220 contains the catalyst that is used in the hydrogenation of the carboxylic acid, preferably acetic acid. During the hydrogenation process, a crude ethanol product is withdrawn, preferably continuously, from reactor 220 via line 256 and directed to separation zone 206.

Separation zone 206 comprises flasher 222, and first column 224. Further columns may be included as need to further separate and purify the crude ethanol product as shown in FIG. 3. The crude ethanol product may be condensed and fed to flasher 222, which, in turn, provides a vapor stream and a liquid stream. Flasher 222 may operate at a temperature from 20° C. to 350° C., e.g., from 30° C. to 325° C. or from 60° C. to 250° C. The pressure of flasher 222 may be from 100 kPa to 3000 kPa, e.g., from 125 kPa to 2500 kPa or from 150 kPa to 2200 kPa.

The vapor stream exiting flasher 222 may comprise hydrogen and hydrocarbons, which may be purged and/or returned to reaction zone 204 via line 258. As shown in FIG. 2, the returned portion of the vapor stream passes through compressor 260 and is combined with the hydrogen feed and co-fed to vaporizer 218.

The liquid from flasher 222 is withdrawn and pumped as a feed composition via line 262 to the side of column 224, which may be referred to as the first column when multiple columns are used as shown in FIG. 3. Column 224 may also be referred to as an “acid separation column.” The contents of line 262 typically will be substantially similar to the product obtained directly from the reactor 220, and may, in fact, also be characterized as a crude ethanol product. However, the feed composition in line 262 preferably has substantially no hydrogen, carbon dioxide, methane or ethane, which are removed by flasher 222. Exemplary compositions of line 262 are provided in Table 2. It should be understood that liquid line 262 may contain other components, not listed, such as additional components in the feed.

TABLE 2
FEED COMPOSITION

| | Conc. (wt. %) | Conc. (wt. %) | Conc. (wt. %) |
|---|---|---|---|
| Ethanol | 5 to 70 | 30 to 70 | 25 to 50 |
| Acetic Acid | <90 | 1 to 80 | 2 to 70 |
| Water | 5 to 60 | 15 to 60 | 20 to 60 |
| Ethyl Acetate | <20 | 0.001 to 15 | 1 to 12 |
| Acetaldehyde | <10 | 0.001 to 3 | 0.1 to 3 |
| Acetal | <5 | 0.001 to 2 | 0.005 to 1 |
| Acetone | <5 | 0.0005 to 0.05 | 0.001 to 0.03 |
| Other Alcohols | <8 | <0.1 | <0.05 |
| Other Esters | <5 | <0.005 | <0.001 |
| Other Ethers | <5 | <0.005 | <0.001 |

The amounts indicated as less than (<) in the tables throughout present application are preferably not present and if present may be present in trace amounts or in amounts greater than 0.0001 wt. %.

The “other esters” in Table 2 may include, but are not limited to, ethyl propionate, methyl acetate, isopropyl acetate, n-propyl acetate, n-butyl acetate or mixtures thereof. The “other ethers” in Table 2 may include, but are not limited to, diethyl ether, methyl ethyl ether, isobutyl ethyl ether or mixtures thereof. The “other alcohols” in Table 3 may include, but are not limited to, methanol, isopropanol, n-propanol, n-butanol or mixtures thereof. In one embodiment, the feed composition, e.g., line 262, may comprise propanol, e.g., isopropanol and/or n-propanol, in an amount from 0.001 to 0.1 wt. %, from 0.001 to 0.05 wt. % or from 0.001 to 0.03 wt. %. It should be understood that these other components may be carried through in any of the distillate or residue streams described herein.

Optionally, the crude ethanol product may pass through one or more membranes to separate hydrogen and/or other non-condensable gases. In other optional embodiments, the crude ethanol product may be fed directly to the acid separation column as a vapor feed and the non-condensable gases may be recovered from the overhead of the column.

When the content of acetic acid in line 262 is less than 5 wt. %, acid separation column 224 may be skipped and line 262 may be introduced directly to a second column, e.g., a “light ends column.” In addition, column 224 may be operated to initially remove a substantial portion of water as the residue.

In the embodiment shown in FIG. 2, line 262 is introduced in the lower part of first column 224, e.g., lower half or lower third. Depending on the acetic acid conversion and operation of column 224, unreacted acetic acid, water, and other heavy components, if present, are removed from the composition in line 262 and are withdrawn, preferably continuously, as residue. In preferred embodiments, the presence of larger amounts of water in line 262 allows separation of a majority of water in line 262 along with substantially all the acetic acid in residue stream 264. All or a portion of residue stream 264 may be recycled to reaction zone 204 as necessary to maintain the water concentration amounts for the acetic acid feed stream. In addition, residue stream 264 may be separated into a water stream and an acetic acid stream, and either stream may be returned to reaction zone 204. In other embodiments, residue stream 264 may be a dilute acid stream that may be treated in a weak acid recovery system or sent to a reactive distillation column to convert the acid to esters.

First column 224 also forms an overhead distillate, which is withdrawn via stream 266, and which may be condensed and refluxed, for example, at a ratio from 10:1 to 1:10, e.g., from 3:1 to 1:3 or from 1:2 to 2:1. As indicated above, a majority of the water is withdrawn in residue via line 264 as opposed to distillate via line 266 such that the weight ratio of water in line 264 to line 266 is greater than 2:1.

The columns shown in the FIGS. may comprise any distillation column capable of performing the desired separation and/or purification. Each column preferably comprises a tray column having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section and so on.

The associated condensers and liquid separation vessels that may be employed with each of the distillation columns may be of any conventional design and are simplified in the FIGS. As shown in the FIGS., heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the FIGS., additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. As a practical matter, pressures from 10 kPa to 3000 kPa will generally be employed in these zones although in some embodiments subatmospheric pressures or superatomic pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

When column 224 is operated under about 170 kPa, the temperature of the residue exiting in line 264 from column 224 preferably is from 90° C. to 130° C., e.g., from 95° C. to 120° C. or from 100° C. to 115° C. The temperature of the distillate exiting in line 266 from column 224 preferably is from 60° C. to 90° C., e.g., from 65° C. to 85° C. or from 70° C. to 80° C. In some embodiments, the pressure of first column 224 may range from 0.1 kPa to 510 kPa, e.g., from 1 kPa to 475 kPa or from 1 kPa to 375 kPa. Distillate and residue compositions for first column 224 for one exemplary embodiment of the present invention are provided in Table 3. In addition, for convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 3
FIRST COLUMN

| | Conc. (wt. %) | Conc. (wt. %) | Conc. (wt. %) |
|---|---|---|---|
| Distillate | | | |
| Ethanol | 20 to 90 | 30 to 85 | 50 to 85 |
| Water | 4 to 38 | 7 to 32 | 7 to 25 |
| Acetic Acid | <1 | 0.001 to 1 | 0.01 to 0.5 |
| Ethyl Acetate | <60 | 5 to 40 | 8 to 45 |
| Acetaldehyde | <10 | 0.001 to 5 | 0.01 to 4 |
| Acetal | <4.0 | <3.0 | <2.0 |
| Acetone | <0.05 | 0.001 to 0.03 | 0.01 to 0.025 |
| Residue | | | |
| Acetic Acid | <90 | 1 to 50 | 2.5 to 40 |
| Water | 30 to 100 | 45 to 90 | 60 to 90 |
| Ethanol | <1 | <0.9 | <0.5 |

As indicated in Table 3, embodiments of the present invention allow a majority of the water to be withdrawn in residue line 264. In addition, the increased amount of water reduces the amount of acetic acid that may be carried over in distillate line 266. Preferably there is substantially no or very low amounts of acetic acid in distillate line 266. Reducing acetic acid in distillate line 266 may advantageously reduce the amount of acetic acid in the final ethanol product.

Some species, such as acetals, may decompose in column 224 such that very low amounts, or even no detectable amounts, of acetals remain in the distillate or residue. In addition, there may be an equilibrium reaction after the crude ethanol product exits reactor 220 in liquid feed 256. Depending on the concentration of acetic acid in the crude ethanol product, equilibrium may be driven toward formation of ethyl acetate. The reaction may be regulated using the residence time and/or temperature of liquid feed 256.

The distillate, e.g., overhead stream, of column 224 optionally is condensed and refluxed as shown in FIG. 2, preferably, at a reflux ratio of 1:5 to 10:1. The distillate in line 266 preferably comprises ethanol, ethyl acetate, and lower amounts of water. The separation of these species may be difficult, in some cases, due to the formation of binary and tertiary azeotropes.

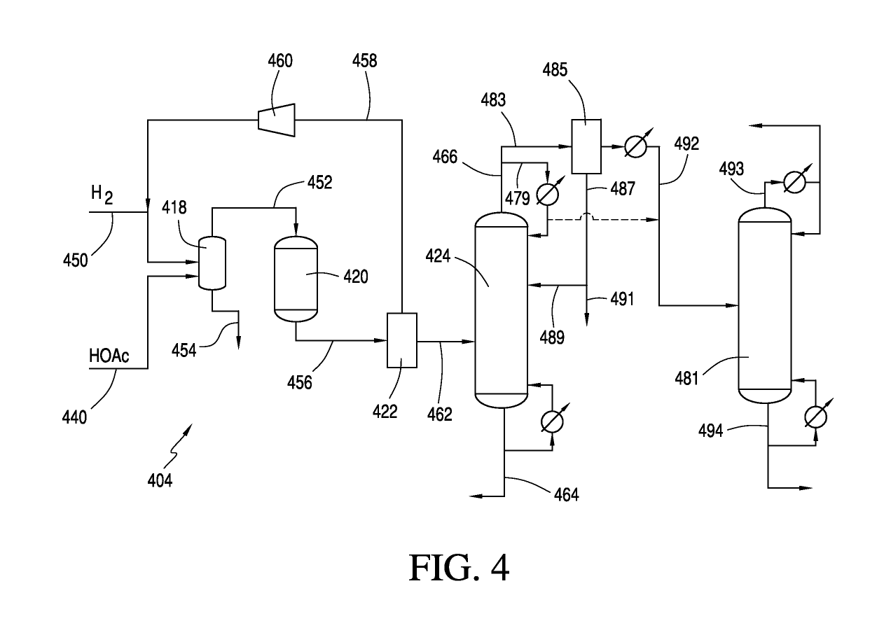

In some embodiments, depending on acetic conversion and the amount of water withdrawn from column 244, distillate in line 266 may comprise a suitable ethanol product that requires no further processing. In one embodiment, distillate in line 266 may be further processed as described in FIG. 4 below.

In one embodiment, as shown in FIG. 3, the liquid stream 362 from flasher 322 is withdrawn and introduced in the lower part of first column 324, e.g., lower half or lower third. First column 324 is also referred to as an “acid separation column.” In one embodiment, the contents of liquid stream 362 are substantially similar to the crude ethanol product obtained from the reactor, except that the composition has been depleted of hydrogen, carbon dioxide, methane and/or ethane, which are removed by flasher 322. Accordingly, liquid stream 362 may also be referred to as a crude ethanol product. Exemplary components of liquid stream 362 are provided in Table 4. It should be understood that liquid stream 362 may contain other components, not listed in Table 4.

TABLE 4
COLUMN FEED COMPOSITION
(Liquid Stream 362)

| | Conc. (wt. %) | Conc. (wt. %) | Conc. (wt. %) |
|---|---|---|---|
| Ethanol | 5 to 70 | 10 to 60 | 15 to 50 |
| Acetic Acid | <90 | 5 to 80 | 5 to 70 |
| Water | 5 to 30 | 5 to 28 | 10 to 26 |
| Ethyl Acetate | <30 | 0.001 to 20 | 1 to 12 |
| Acetaldehyde | <10 | 0.001 to 3 | 0.1 to 3 |
| Diethyl Acetal | 0.01 to 10 | 0.01 to 6 | 0.01 to 5 |
| Acetone | <5 | 0.0005 to 0.05 | 0.001 to 0.03 |
| Other Esters | <5 | <0.005 | <0.001 |
| Other Ethers | <5 | <0.005 | <0.001 |
| Other Alcohols | <5 | <0.005 | <0.001 |

The amounts indicated as less than (<) in the tables throughout present specification are preferably not present and if present may be present in trace amounts or in amounts greater than 0.0001 wt. %.

The “other esters” in Table 4 may include, but are not limited to, ethyl propionate, methyl acetate, isopropyl acetate, n-propyl acetate, n-butyl acetate or mixtures thereof. The “other ethers” in Table 4 may include, but are not limited to, diethyl ether, methyl ethyl ether, isobutyl ethyl ether or mixtures thereof. The “other alcohols” in Table 4 may include, but are not limited to, methanol, isopropanol, n-propanol, n-butanol or mixtures thereof. In one embodiment, the liquid stream 362 may comprise propanol, e.g., isopropanol and/or n-propanol, in an amount from 0.001 to 0.1 wt. %, from 0.001 to 0.05 wt. % or from 0.001 to 0.03 wt. %. In should be understood that these other components may be carried through in any of the distillate or residue streams described herein and will not be further described herein, unless indicated otherwise.

Optionally, crude ethanol product in line 356 or in liquid stream 362 may be further fed to an esterification reactor, hydrogenolysis reactor, or combination thereof. An esterification reactor may be used to consume residual acetic acid present in the crude ethanol product to further reduce the amount of acetic acid that would otherwise need to be removed. Hydrogenolysis may be used to convert ethyl acetate in the crude ethanol product to ethanol.

In the embodiment shown in FIG. 3, line 362 is introduced in the lower part of first column 324, e.g., lower half or lower third. In first column 324, unreacted acetic acid, a portion of the water, and other heavy components, if present, are removed from the composition in line 364 and are withdrawn, preferably continuously, as residue. Some or all of the residue may be returned and/or recycled back to reaction zone 304 (not shown). Recycling the acetic acid in line 364 to the vaporizer 318 may reduce the amount of heavies that need to be purged from vaporizer 318. Reducing the amount of heavies to be purged may improve efficiencies of the process while reducing byproducts.

First column 324 also forms an overhead distillate, which is withdrawn in line 366, and which may be condensed and refluxed, for example, at a ratio from 10:1 to 1:10, e.g., from 3:1 to 1:3 or from 1:2 to 2:1.

When first column 324 is operated under standard atmospheric pressure, the temperature of the residue exiting in line 364 preferably is from 95° C. to 120° C., e.g., from 110° C. to 117° C. or from 111° C. to 115° C. The temperature of the distillate exiting in line 366 preferably is from 70° C. to 110° C., e.g., from 75° C. to 95° C. or from 80° C. to 90° C. Column 324 preferably operates at ambient pressure. In other embodiments, the pressure of first column 324 may range from 0.1 kPa to 510 kPa, e.g., from 1 kPa to 475 kPa or from 1 kPa to 375 kPa. Exemplary components of the distillate and residue compositions for first column 324 are provided in Table 5 below. It should also be understood that the distillate and residue may also contain other components, not listed, such as components in the feed. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 5
ACID COLUMN 324 (FIG. 3)

| | Conc. (wt. %) | Conc. (wt. %) | Conc. (wt. %) |
|---|---|---|---|
| Distillate | | | |
| Ethanol | 20 to 75 | 30 to 70 | 40 to 65 |
| Water | 10 to 40 | 15 to 35 | 20 to 35 |
| Acetic Acid | <2 | 0.001 to 0.5 | 0.01 to 0.2 |
| Ethyl Acetate | <60 | 5.0 to 40 | 10 to 30 |
| Acetaldehyde | <10 | 0.001 to 5 | 0.01 to 4 |
| Diethyl Acetal | 0.01 to 10 | 0.05 to 6 | 0.1 to 5 |
| Acetone | <0.05 | 0.001 to 0.03 | 0.01 to 0.025 |
| Residue | | | |
| Acetic Acid | 60 to 100 | 70 to 95 | 85 to 92 |
| Water | <30 | 1 to 20 | 1 to 15 |
| Ethanol | <1 | <0.9 | <0.07 |

As shown in Table 5, without being bound by theory, it has surprisingly and unexpectedly been discovered that when any amount of acetal is detected in the feed that is introduced to the acid separation column 324, the acetal appears to decompose in the column such that less or even no detectable amounts are present in the distillate and/or residue.

The distillate in line 366 preferably comprises ethanol, ethyl acetate, and water, along with other impurities, which may be difficult to separate due to the formation of binary and tertiary azeotropes. To further separate distillate, line 366 is introduced to the second column 368, also referred to as the “light ends column,” preferably in the middle part of column 368, e.g., middle half or middle third. Preferably the second column 368 is an extractive distillation column, and an extraction agent is added thereto. Extractive distillation is a method of separating close boiling components, such as azeotropes, by distilling the feed in the presence of an extraction agent. The extraction agent preferably has a boiling point that is higher than the compounds being separated in the feed. In preferred embodiments, the extraction agent is comprised primarily of water. As indicated above, the first distillate in line 366 that is fed to the second column 368 comprises ethyl acetate, ethanol, and water. These compounds tend to form binary and ternary azeotropes, which decrease separation efficiency. As shown, in one embodiment the extraction agent comprises a third residue from a third column. Preferably, the recycled third residue is fed to second column 368 at a point higher than the first distillate in line 366 is fed. In one embodiment, the recycled third residue is fed near the top of second column 368 or fed, for example, above the feed in line 366 and below the reflux line from the condensed overheads. In a tray column, the third residue is continuously added near the top of the second column 368 so that an appreciable amount of the third residue is present in the liquid phase on all of the trays below. In another embodiment, the extraction agent is fed from a source outside of the process 300 to second column 368. Preferably this extraction agent comprises water.

The molar ratio of the water in the extraction agent to the ethanol in the feed to the second column is preferably at least 0.5:1, e.g., at least 1:1 or at least 3:1. In terms of ranges, preferred molar ratios may range from 0.5:1 to 8:1, e.g., from 1:1 to 7:1 or from 2:1 to 6.5:1. Higher molar ratios may be used but with diminishing returns in terms of the additional ethyl acetate in the second distillate and decreased ethanol concentrations in the second column distillate.

In one embodiment, an additional extraction agent, such as water from an external source, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane and chlorinated paraffins, may be added to second column 123. Some suitable extraction agents include those described in U.S. Pat. Nos. 4,379,028, 4,569,726, 5,993,610 and 6,375,807, the entire contents and disclosure of which are hereby incorporated by reference. The additional extraction agent may be combined with the recycled third residue and co-fed to second column 368. The additional extraction agent may also be added separately to the second column 368. In one aspect, the extraction agent comprises an extraction agent, e.g., water, derived from an external source and none of the extraction agent is derived from the third residue.

In the embodiments of the present invention, without the use of an extractive agent, a larger portion of the ethanol would carry over into the second distillate in line 372. By using an extractive agent in second column 368, the separation of ethanol into the second residue in line 370 is facilitated thus increasing the yield of the overall ethanol product in the second residue in line 370.

Second column 368 may be a tray or packed column. In one embodiment, second column 368 is a tray column having from 5 to 70 trays, e.g., from 15 to 50 trays or from 20 to 45 trays. Although the temperature and pressure of second column 368 may vary, when at atmospheric pressure the temperature of the second residue exiting in line 370 preferably is from 60° C. to 90° C., e.g., from 70° C. to 90° C. or from 80° C. to 90° C. The temperature of the second distillate exiting in line 372 from second column 368 preferably is from 50° C. to 90° C., e.g., from 60° C. to 80° C. or from 60° C. to 70° C. Column 368 may operate at atmospheric pressure. In other embodiments, the pressure of second column 368 may range from 0.1 kPa to 510 kPa, e.g., from 1 kPa to 475 kPa or from 1 kPa to 375 kPa. Exemplary components for the distillate and residue compositions for second column 368 are provided in Table 6 below. It should be understood that the distillate and residue may also contain other components, not listed, such as components in the feed.

TABLE 6
SECOND COLUMN 368 (FIG. 3)

| | Conc. (wt. %) | Conc. (wt. %) | Conc. (wt. %) |
|---|---|---|---|
| Distillate | | | |
| Ethyl Acetate | 10 to 99 | 25 to 95 | 50 to 93 |
| Acetaldehyde | <25 | 0.5 to 15 | 1 to 8 |
| Water | <25 | 0.5 to 20 | 4 to 16 |
| Ethanol | <30 | 0.001 to 15 | 0.01 to 5 |
| Diethyl Acetal | 0.01 to 20 | 1 to 20 | 5 to 20 |
| Residue | | | |
| Water | 30 to 90 | 40 to 85 | 50 to 85 |
| Ethanol | 10 to 75 | 15 to 60 | 20 to 50 |
| Ethyl Acetate | <3 | 0.001 to 2 | 0.001 to 0.5 |
| Acetic Acid | <0.5 | 0.001 to 0.3 | 0.001 to 0.2 |

In preferred embodiments, the recycling of the third residue promotes the separation of ethyl acetate from the residue of the second column 368. For example, the weight ratio of ethyl acetate in the second residue to second distillate preferably is less than 0.4:1, e.g., less than 0.2:1 or less than 0.1:1. In embodiments that use an extractive distillation column with water as an extraction agent as the second column 368, the weight ratio of ethyl acetate in the second residue to ethyl acetate in the second distillate approaches zero. Second residue may comprise, for example, from 30% to 99.5% of the water and from 85 to 100% of the acetic acid from line 366. The second distillate in line 372 comprises ethyl acetate and DEA and additionally comprises water, ethanol, and/or acetaldehyde. Second distillate 372 is preferably substantially free of acetic acid. At least a portion of the second distillate is returned to reaction zone 304. For example, the second distillate may be combined with the acetic acid feed, added to vaporizer 318, or added directly to reactor 320. The second distillate preferably is co-fed with the acetic acid in feed line 340 to vaporizer 318. Preferably, conversion of DEA in reactor 320 is at least 50%, e.g., 60% or 70%.

The weight ratio of ethanol in the second residue to second distillate preferably is at least 3:1, e.g., at least 6:1, at least 8:1, at least 10:1 or at least 15:1. All or a portion of the third residue is recycled to second column 368. In one embodiment, all of the third residue may be recycled until process 300 reaches a steady state and then a portion of the third residue is recycled with the remaining portion being purged from the process 300. The composition of the second residue will tend to have lower amounts of ethanol than when the third residue is not recycled. As the third residue is recycled, the composition of the second residue, as provided in Table 7, comprises less than 30 wt. % of ethanol, e.g., less than 20 wt. % or less than 15 wt. %. The majority of the second residue preferably comprises water. Notwithstanding this effect, the extractive distillation step advantageously also reduces the amount of ethyl acetate that is sent to the third column, which is highly beneficial in ultimately forming a highly pure ethanol product.

As shown, the second residue from second column 368, which comprises ethanol and water, is fed via line 370 to third column 376, also referred to as the “product column.” More preferably, the second residue in line 370 is introduced in the lower part of third column 376, e.g., lower half or lower third. Third column 376 recovers ethanol, which preferably is substantially pure with respect to organic impurities and other than the azeotropic water content, as the distillate in line 378. The distillate of third column 376 preferably is refluxed as shown in FIG. 3, for example, at a reflux ratio from 1:10 to 10:1, e.g., from 1:3 to 3:1 or from 1:2 to 2:1. The third residue in line 380, which comprises primarily water, preferably is returned to the second column 368 as an extraction agent as described above. In one embodiment, a first portion of the third residue in line 380 is recycled to the second column and a second portion is purged and removed from the system. In one embodiment, once the process reaches steady state, the second portion of water to be purged is substantially similar to the amount water formed in the hydrogenation of acetic acid. In one embodiment, a portion of the third residue may be used to hydrolyze any other stream, such as one or more streams comprising ethyl acetate.

Although third residue may be directly recycled to second column 368, third residue may also be returned indirectly, for example, by storing a portion or all of the third residue in a tank (not shown) or treating the third residue to further separate any minor components such as aldehydes, higher molecular weight alcohols, or esters in one or more additional columns (not shown).

Third column 376 is preferably a tray column as described above and operates at atmospheric pressure or optionally at pressures above or below atmospheric pressure. The temperature of the third distillate exiting in line 378 preferably is from 50° C. to 110° C., e.g., from 70° C. to 100° C. or from 75° C. to 95° C. The temperature of the third residue in line 380 preferably is from 15° C. to 100° C., e.g., from 30° C. to 90° C. or from 50° C. to 80° C. Exemplary components of the distillate and residue compositions for third column 376 are provided in Table 7 below. It should be understood that the distillate and residue may also contain other components, not listed, such as components in the feed.

TABLE 7
THIRD COLUMN 376 (FIG. 3)

| | Conc. (wt. %) | Conc. (wt. %) | Conc. (wt. %) |
|---|---|---|---|
| Distillate | | | |
| Ethanol | 75 to 96 | 80 to 96 | 85 to 96 |
| Water | <12 | 1 to 9 | 3 to 8 |
| Acetic Acid | <12 | 0.0001 to 0.1 | 0.005 to 0.05 |
| Ethyl Acetate | <12 | 0.0001 to 0.05 | 0.005 to 0.025 |
| Acetaldehyde | <12 | 0.0001 to 0.1 | 0.005 to 0.05 |
| Diethyl Acetal | <12 | 0.0001 to 0.05 | 0.005 to 0.01 |
| Residue | | | |
| Water | 75 to 100 | 80 to 100 | 90 to 100 |
| Ethanol | <0.8 | 0.001 to 0.5 | 0.005 to 0.05 |
| Ethyl Acetate | <1 | 0.001 to 0.5 | 0.005 to 0.2 |
| Acetic Acid | <2 | 0.001 to 0.5 | 0.005 to 0.2 |

In one embodiment, the third residue in line 380 is withdrawn from third column 376 at a temperature higher than the operating temperature of the second column 368. Preferably, the third residue in line 380 is integrated to heat one or more other streams or is reboiled prior to be returned to the second column 368.

Any of the compounds that are carried through the distillation process from the feed or crude reaction product generally remain in the third distillate in amounts of less 0.01 wt. %, based on the total weight of the third distillate composition, e.g., less than 0.05 wt. % or less than 0.02 wt. %. In one embodiment, one or more side streams may remove impurities, especially those impurities from the methanol feed to the carbonylation process, from any of the columns in the process 300. Preferably at least one side stream is used to remove impurities from the third column 376. The impurities may be purged and/or retained within process 300.

The third distillate in line 378 may be further purified to form an anhydrous ethanol product stream, i.e., “finished anhydrous ethanol,” using one or more additional separation systems, such as, for example, distillation columns, adsorption units, membranes, or molecular sieves. Suitable adsorption units include pressure swing adsorption units and thermal swing adsorption unit. Preferably, third distillate comprises less than 0.01 wt. % DEA, e.g., less than 0.005 wt. % or less than 0.003 wt. %.

Returning to second column 368, the second distillate preferably is refluxed as shown in FIG. 3, optionally at a reflux ratio of 1:10 to 10:1, e.g., from 1:5 to 5:1 or from 1:3 to 3:1. As explained above, at least a portion of second distillate in line 372 may be purged or recycled to the reaction zone. In an optional embodiment, at least a portion of second distillate in line 372 is further processed in an optional fourth column 374, also referred to as the “acetaldehyde removal column.” In fourth column 374, the second distillate is separated into a fourth distillate, which comprises acetaldehyde and DEA, in line 382 and a fourth residue, which comprises ethyl acetate, in line 384. The fourth distillate preferably is refluxed at a reflux ratio from 1:20 to 20:1, e.g., from 1:15 to 15:1 or from 1:10 to 10:1, and at least a portion of the fourth distillate is returned to reaction zone 304. For example, the fourth distillate may be combined with the acetic acid feed, added to vaporizer 318, or added directly to reactor 320. The fourth distillate preferably is co-fed with the acetic acid in feed line 340 to vaporizer 318. Without being bound by theory, since acetaldehyde and DEA may be reacted, e.g., by hydrogenation, to form ethanol, the recycling of a stream that contains acetaldehyde and DEA to the reaction zone increases the yield of ethanol and decreases byproduct and waste generation. DEA may be present in fourth distillate in an amount from 0.01 to 20 wt. %, e.g., from 1 to 20 wt. % or from 5 to 20 wt. %. In another embodiment, the acetaldehyde may be collected and utilized, with or without further purification, to make useful products including but not limited to n-butanol, 1,3-butanediol, and/or crotonaldehyde and derivatives.

The fourth residue of optional fourth column 374 may be purged. The fourth residue primarily comprises ethyl acetate and ethanol, which may be suitable for use as a solvent mixture or in the production of esters. In one preferred embodiment, the acetaldehyde is removed from the second distillate in optional fourth column 374 such that no detectable amount of acetaldehyde is present in the residue of column 374.

Optional fourth column 374 is a tray column as described above and may operate above atmospheric pressure. In one embodiment, the pressure is from 120 kPa to 5,000 kPa, e.g., from 200 kPa to 4,500 kPa, or from 400 kPa to 3,000 kPa. In a preferred embodiment the fourth column 374 may operate at a pressure that is higher than the pressure of the other columns.

The temperature of the fourth distillate exiting in line 382 preferably is from 60° C. to 110° C., e.g., from 70° C. to 100° C. or from 75° C. to 95° C. The temperature of the residue in line 384 preferably is from 70° C. to 115° C., e.g., from 80° C. to 110° C. or from 85° C. to 110° C. Exemplary components of the distillate and residue compositions for optional fourth column 374 are provided in Table 8 below. It should be understood that the distillate and residue may also contain other components, not listed, such as components in the feed.

TABLE 8
OPTIONAL FOURTH COLUMN 374 (FIG. 3)

| | Conc. (wt. %) | Conc. (wt. %) | Conc. (wt. %) |
|---|---|---|---|
| Distillate | | | |
| Acetaldehyde | 2 to 80 | 2 to 50 | 5 to 40 |
| Ethyl Acetate | <90 | 30 to 80 | 40 to 75 |
| Ethanol | <30 | 0.001 to 25 | 0.01 to 20 |
| Water | <25 | 0.001 to 20 | 0.01 to 15 |
| Diethyl Acetal | 0.01 to 20 | 1 to 20 | 5 to 20 |
| Residue | | | |
| Ethyl Acetate | 40 to 100 | 50 to 100 | 60 to 100 |
| Ethanol | <40 | 0.001 to 30 | 0.01 to 15 |
| Water | <25 | 0.001 to 20 | 2 to 15 |
| Acetaldehyde | <1 | 0.001 to 0.5 | Not detectable |
| Diethyl Acetal | <3 | 0.0001 to 2 | 0.001 to 0.01 |

In one embodiment, a portion of the third residue in line 384 is recycled to second column 368. In one embodiment, recycling the third residue further reduces the aldehyde components in the second residue and concentrates these aldehyde components in second distillate in line 372 and thereby sent to optional fourth column 374, wherein the aldehydes may be more easily separated. The third distillate in line 378 may have lower concentrations of aldehydes and esters due to the recycling of third residue in line 380. Preferably, the third distillate in line 378 has less than 0.01 wt. % DEA.

FIG. 4 illustrates another exemplary separation system in which distillate in line 262 from FIG. 2 is fed to a water separator and an additional column.

The reaction zone 404 of FIG. 4 is similar to that of FIG. 2 and similar numbers indicate similar items. Reaction zone 404 produces liquid feed 462. In one preferred embodiment, reaction zone 404 of FIG. 4 operates at above 80% acetic acid conversion, e.g., above 90% conversion or above 99% conversion. Thus, the acetic acid concentration in the liquid feed 462 may be low.

The first distillate in line 466 comprises water, in addition to ethanol and other organics. In terms of ranges, the concentration of water in the first distillate in line 466 preferably is from 4 wt. % to 38 wt. %, e.g., from 7 wt. % to 32 wt. %, or from 7 to 25 wt. %. A portion of first distillate in line 479 may be condensed and refluxed, for example, at a ratio from 10:1 to 1:10, e.g., from 3:1 to 1:3 or from 1:2 to 2:1. It is understood that reflux ratios may vary with the number of stages, feed locations, column efficiency and/or feed composition. Operating with a reflux ratio of greater than 3:1 may be less preferred because more energy may be required to operate first column 424. The condensed portion of the first distillate may also be fed to second column 481.

The remaining portion of the first distillate in line 483 is fed to a water separation unit 485. Water separation unit 485 may be an adsorption unit, membrane, molecular sieves, extractive column distillation, or a combination thereof. A membrane or an array of membranes may also be employed to separate water from the distillate. The membrane or array of membranes may be selected from any suitable membrane that is capable of removing a permeate water stream from a stream that also comprises ethanol and ethyl acetate.

In a preferred embodiment, water separator 485 is a pressure swing adsorption (PSA) unit. The PSA unit is optionally operated at a temperature from 30° C. to 160° C., e.g., from 80° C. to 140° C., and a pressure from 0.01 kPa to 550 kPa, e.g., from 1 kPa to 150 kPa. The PSA unit may comprise two to five beds. Water separator 485 may remove at least 95% of the water from the portion of first distillate in line 483, and more preferably from 95% to 99.99% of the water from the first distillate, in a water stream 487. All or a portion of water stream 487 may be returned to column 424 in line 489, where the water preferably is ultimately recovered from column 424 in the first residue in line 464. Additionally or alternatively, all or a portion of water stream 487 may be purged via line 491. The remaining portion of first distillate exits the water separator 485 as ethanol mixture stream 492. Ethanol mixture stream 492 may have a low concentration of water of less than 10 wt. %, e.g., less than 6 wt. % or less than 2 wt. %. Exemplary components of ethanol mixture stream 492 and first residue in line 464 are provided in Table 9 below. It should also be understood that these streams may also contain other components, not listed, such as components derived from the feed.

TABLE 9
FIRST COLUMN WITH PSA

| | Conc. (wt. %) | Conc. (wt. %) | Conc. (wt. %) |
|---|---|---|---|
| Ethanol Mixture | | | |
| Stream | | | |
| Ethanol | 20 to 95 | 30 to 95 | 40 to 95 |
| Water | <10 | 0.01 to 6 | 0.1 to 2 |
| Acetic Acid | <2 | 0.001 to 0.5 | 0.01 to 0.2 |
| Ethyl Acetate | <60 | 1 to 55 | 5 to 55 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | <90 | 1 to 50 | 2 to 35 |
| Water | 30 to 100 | 45 to 95 | 60 to 90 |
| Ethanol | <1 | <0.9 | <0.3 |

Preferably, ethanol mixture stream 492 is not returned or refluxed to first column 424. The condensed portion of the first distillate in line 479 may be combined with ethanol mixture stream 492 to control the water concentration fed to second column 481. For example, in some embodiments the first distillate may be split into equal portions, while in other embodiments, all of the first distillate may be condensed or all of the first distillate may be processed in the water separation unit. In FIG. 4, the condensed portion in line 479 and ethanol mixture stream 492 are co-fed to second column 481. In other embodiments, the condensed portion in line 479 and ethanol mixture stream 492 may be separately fed to second column 481. The combined distillate and ethanol mixture has a total water concentration of greater than 0.5 wt. %, e.g., greater than 2 wt. % or greater than 5 wt. %. In terms of ranges, the total water concentration of the combined distillate and ethanol mixture may be from 0.5 to 15 wt. %, e.g., from 2 to 12 wt. %, or from 5 to 10 wt. %.

Second column 481 in FIG. 4, also referred to as the “light ends column,” removes ethyl acetate and acetaldehyde from the first distillate in line 479 and/or ethanol mixture stream 492. Ethyl acetate and acetaldehyde are removed as a second distillate in line 493 and ethanol is removed as the second residue in line 494. Second column 481 may be a tray column or packed column. In one embodiment, second column 481 is a tray column having from 5 to 70 trays, e.g., from 15 to 50 trays or from 20 to 45 trays.

Second column 481 operates at a pressure ranging from 0.1 kPa to 510 kPa, e.g., from 10 kPa to 450 kPa or from 50 kPa to 350 kPa. Although the temperature of second column 481 may vary, when at about 20 kPa to 70 kPa, the temperature of the second residue exiting in line 494 preferably is from 30° C. to 75° C., e.g., from 35° C. to 70° C. or from 40° C. to 65° C. The temperature of the second distillate exiting in line 493 preferably is from 20° C. to 55° C., e.g., from 25° C. to 50° C. or from 30° C. to 45° C.

The total concentration of water fed to second column 481 preferably is less than 10 wt. %, as discussed above. When first distillate in line 479 and/or ethanol mixture stream 492 comprises minor amounts of water, e.g., less than 1 wt. % or less than 0.5 wt. %, additional water may be fed to the second column 481 as an extractive agent in the upper portion of the column. A sufficient amount of water is preferably added via the extractive agent such that the total concentration of water fed to second column 481 is from 1 to 10 wt. % water, e.g., from 2 to 6 wt. %, based on the total weight of all components fed to second column 481. If the extractive agent comprises water, the water may be obtained from an external source or from an internal return/recycle line from one or more of the other columns or water separators.

Suitable extractive agents may also include, for example, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane, chlorinated paraffins, or a combination thereof. When extractive agents are used, a suitable recovery system, such as a further distillation column, may be used to recycle the extractive agent.

Exemplary components for the second distillate and second residue compositions for the second column 481 are provided in Table 10, below. It should be understood that the distillate and residue may also contain other components, not listed in Table 10.

TABLE 10
SECOND COLUMN (FIG. 4)

| | Conc. (wt. %) | Conc. (wt. %) | Conc. (wt. %) |
|---|---|---|---|
| Second Distillate | | | |
| Ethyl Acetate | 5 to 90 | 10 to 80 | 15 to 75 |
| Acetaldehyde | <60 | 1 to 40 | 1 to 35 |
| Ethanol | <45 | 0.001 to 40 | 0.01 to 35 |
| Water | <20 | 0.01 to 10 | 0.1 to 5 |
| Second Residue | | | |
| Ethanol | 80 to 99.5 | 85 to 97 | 60 to 95 |
| Water | <20 | 0.001 to 15 | 0.01 to 10 |
| Ethyl Acetate | <1 | 0.001 to 2 | 0.001 to 0.5 |
| Acetic Acid | <0.5 | <0.01 | 0.001 to 0.01 |

The second residue in FIG. 4 comprises one or more impurities selected from the group consisting of ethyl acetate, acetic acid, acetaldehyde, and diethyl acetal. The second residue may comprise at least 100 wppm of these impurities, e.g., at least 250 wppm or at least 500 wppm. In some embodiments, the second residue may contain substantially no ethyl acetate or acetaldehyde.

The second distillate in line 493, which comprises ethyl acetate and/or acetaldehyde, preferably is refluxed as shown in FIG. 4, for example, at a reflux ratio from 1:30 to 30:1, e.g., from 1:10 to 10:1 or from 1:3 to 3:1. In one aspect, not shown, the second distillate 493 or a portion thereof may be returned to reactor 408. The ethyl acetate and/or acetaldehyde in the second distillate may be further reacted in reactor 408.

In one embodiment, the second distillate in line 493 and/or a refined second distillate, or a portion of either or both streams, may be further separated to produce an acetaldehyde-containing stream and an ethyl acetate-containing stream. This may allow a portion of either the resulting acetaldehyde-containing stream or ethyl acetate-containing stream to be recycled to reactor 408 while purging the other stream. The purge stream may be valuable as a source of either ethyl acetate and/or acetaldehyde.

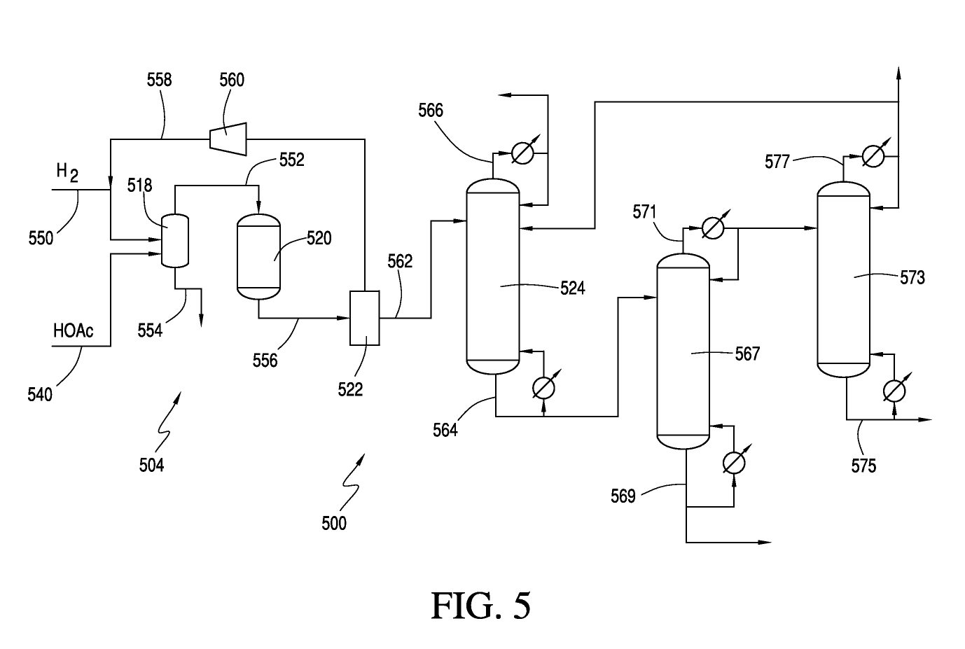

FIG. 5 shows another exemplary separation system. The reaction zone 504 of FIG. 5 is similar to that of FIGS. 3 and 4 and similar numbers indicate similar items. Reaction zone 504 produces liquid feed 562, for further separation. In one preferred embodiment, reaction zone 504 of FIG. 5 operates at above 80% acetic acid conversion, e.g., above 90% conversion or above 99% conversion. Thus, the acetic acid concentration in the liquid stream 562 may be low.

In the exemplary embodiment shown in FIG. 5, liquid stream 562 is introduced in the upper part of first column 524, e.g., upper half or upper third. In addition to liquid stream 562, an optional extractive agent (not shown) and an optional ethyl acetate recycle stream in line 577 may also be fed to first column 524. The optional extractive agent may comprise water that is introduced above the feed location of the liquid stream 562. In some embodiment, the optional extractive agent may be a dilute acid stream comprising up to 20 wt. % acetic acid. Also, the optional ethyl acetate recycle stream may have a relatively high ethanol concentration, e.g. from 70 to 90 wt. %, and may be fed above or near the feed point of the liquid stream 562.

In one embodiment, first column 524 is a tray column having from 5 to 90 theoretical trays, e.g. from 10 to 60 theoretical trays or from 15 to 50 theoretical trays. The number of actual trays for each column may vary depending on the tray efficiency, which is typically from 0.5 to 0.7 depending on the type of tray. The trays may be sieve trays, fixed valve trays, movable valve trays, or any other suitable design known in the art. In other embodiments, a packed column having structured packing or random packing may be employed.

When first column 524 is operated under 50 kPa, the temperature of the residue exiting in line 564 preferably is from 20° C. to 100° C., e.g., from 30° C. to 90° C. or from 40° C. to 80° C. The base of column 524 may be maintained at a relatively low temperature by withdrawing a residue stream comprising ethanol, ethyl acetate, water, and acetic acid, thereby providing an energy efficiency advantage. The temperature of the distillate exiting in line 566 preferably at 50 kPa is from 10° C. to 80° C., e.g., from 20° C. to 70° C. or from 30° C. to 60° C. The pressure of first column 524 may range from 0.1 kPa to 510 kPa, e.g., from 1 kPa to 475 kPa or from 1 kPa to 375 kPa. In some embodiments, first column 524 may operate under a vacuum of less than 70 kPa, e.g., less than 50 kPa, or less than 20 kPa. Operating under a vacuum may decrease the reboiler duty and reflux ratio of first column 524. However, a decrease in operating pressure for first column 524 does not substantially affect column diameter.

In first column 524, a weight majority of the ethanol, water, acetic acid, are removed from an organic feed, which comprises liquid stream 562 and the optional ethyl acetate recycle stream in line 577, and are withdrawn, preferably continuously, as residue in line 564. This includes any water added as the optional extractive agent. Concentrating the ethanol in the residue reduces the amount of ethanol that is recycled to reactor 520 and in turn reduces the size of reactor 520. Preferably less than 10% of the ethanol from the organic feed, e.g., less than 5% or less than 1% of the ethanol, is returned to reactor 520 from first column 524. In addition, concentrating the ethanol also will concentrate the water and/or acetic acid in the residue. In one embodiment, at least 90% of the ethanol from the organic feed is withdrawn in the residue, and more preferably at least 95%. In addition, ethyl acetate may also be present in the first residue in line 564. The reboiler duty may decrease with an ethyl acetate concentration increase in the first residue in line 564.

First column 524 also forms a distillate, which is withdrawn in line 566, and which may be condensed and refluxed, for example, at a ratio from 30:1 to 1:30, e.g., from 10:1 to 1:10 or from 5:1 to 1:5. Higher mass flow ratios of water to organic feed may allow first column 524 to operate with a reduced reflux ratio.

First distillate in line 566 preferably comprises a weight majority of the acetaldehyde and ethyl acetate from liquid stream 562, as well as from the optional ethyl acetate recycle stream in line 577. In one embodiment, the first distillate in line 566 comprises a concentration of ethyl acetate that is less than the ethyl acetate concentration for the azeotrope of ethyl acetate and water, and more preferably less than 75 wt. %.

In some embodiments, first distillate in stream 566 also comprises ethanol. Returning the first distillate comprising ethanol to the reactor may require an increase in reactor capacity to maintain the same level of ethanol efficiency. In one embodiment, it is preferred to return to the reactor less than 10% of the ethanol from the crude ethanol stream, e.g., less than 5% or less than 1%. In terms of ranges, the amount of returned ethanol is from 0.01 to 10% of the ethanol in the crude ethanol stream, e.g. from 0.1 to 5% or from 0.2 to 1%. In one embodiment, to reduce the amount of ethanol returned, the ethanol may be recovered from the first distillate in line 566 using an optional extractor or extractive distillation column.

Exemplary components of the distillate and residue compositions for first column 524 are provided in Table 11 below. It should also be understood that the distillate and residue may also contain other components, not listed in Table 11. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 11
FIRST COLUMN (FIG. 5)

| | Conc. (wt. %) | Conc. (wt. %) | Conc. (wt. %) |
|---|---|---|---|
| Distillate | | | |
| Ethyl Acetate | 10 to 85 | 15 to 80 | 20 to 75 |
| Acetaldehyde | 0.1 to 70 | 0.2 to 65 | 0.5 to 65 |
| Acetal | <0.1 | <0.1 | <0.05 |
| Acetone | <0.05 | 0.001 to 0.03 | 0.01 to 0.025 |
| Ethanol | 3 to 55 | 4 to 50 | 5 to 45 |
| Water | 0.1 to 20 | 1 to 15 | 2 to 10 |
| Acetic Acid | <2 | <0.1 | <0.05 |
| Residue | | | |
| Acetic Acid | 0.1 to 50 | 0.5 to 40 | 1 to 30 |
| Water | 5 to 40 | 5 to 35 | 10 to 25 |
| Ethanol | 10 to 75 | 15 to 70 | 20 to 65 |
| Ethyl Acetate | 0.005 to 30 | 0.03 to 25 | 0.08 to 1 |

In an embodiment of the present invention, column 524 may be operated at a temperature where most of the water, ethanol, and acetic acid are removed into the residue stream and only a small amount of ethanol and water is collected in the distillate stream due to the formation of binary and tertiary azeotropes. The weight ratio of water in the residue in line 564 to water in the distillate in line 566 may be greater than 1:1, e.g., greater than 2:1. The weight ratio of ethanol in the residue to ethanol in the distillate may be greater than 1:1, e.g., greater than 2:1

The amount of acetic acid in the first residue may vary depending primarily on the conversion in reactor 520. In one embodiment, when the conversion is high, e.g., greater than 90%, the amount of acetic acid in the first residue may be less than 10 wt. %, e.g., less than 5 wt. % or less than 2 wt. %. In other embodiments, when the conversion is lower, e.g., less than 90%, the amount of acetic acid in the first residue may be greater than 10 wt. %.

The distillate preferably is substantially free of acetic acid, e.g., comprising less than 1000 ppm, less than 500 ppm or less than 100 ppm acetic acid. The distillate may be purged from the system or recycled in whole or part to reactor 520. In some embodiments, the distillate may be further separated, e.g., in a distillation column (not shown), into an acetaldehyde stream and an ethyl acetate stream. Either of these streams may be returned to reactor 520 or separated from system 500 as additional product. The ethyl acetate stream may also be hydrolyzed or reduced with hydrogen, via hydrogenolysis, to produce ethanol. When additional ethanol is produced, it is preferred that the additional ethanol is recovered and not directed to reactor 520.

Some species, such as acetals, may decompose in first column 524 such that very low amounts, or even no detectable amounts, of acetals remain in the distillate or residue.

To recover ethanol, first residue in line 564 may be further separated depending on the concentration of acetic acid and/or ethyl acetate. In FIG. 5, residue in line 564 is further separated in a second column 567, also referred to as an “acid column.” Second column 567 yields a second residue in line 569 comprising acetic acid and water, and a second distillate in line 571 comprising ethanol and ethyl acetate. In one embodiment, a weight majority of the water and/or acetic acid fed to second column 567 is removed in the second residue in line 569, e.g., at least 60% of the water and/or acetic acid is removed in the second residue in line 569 or more preferably at least 80% of the water and/or acetic acid. An acid column may be desirable, for example, when the acetic acid concentration in the first residue is greater 50 wppm, e.g., greater than 0.1 wt. %, greater than 1 wt. %, e.g., greater than 5 wt. %.

In one embodiment, a portion of the first residue in line 564 may be preheated prior to being introduced into second column 567, as shown in FIG. 5. After preheating, first residue in line 564 may be converted into a partial vapor feed having less than 30 mol. % of the contents in the vapor phase, e.g., less than 25 mol. % or less than 20 mol. %. In terms of ranges, from 1 to 30 mol. % is in the vapor phase, e.g., from 5 to 20 mol. %. Greater vapor phase contents result in increased energy consumption and a significant increase in the size of second column 567.

Second column 567 operates in a manner to concentrate the ethanol from first residue so that a majority of the ethanol is carried overhead. Thus, the residue of second column 569 may have a low ethanol concentration of less than 5 wt. %, e.g. less than 1 wt. % or less than 0.5 wt. %. Lower ethanol concentrations may be achieved without significant increases in reboiler duty or column size. Thus, in some embodiments, it is efficient to reduce the ethanol concentration in the residue to less than 50 wppm, or more preferably less than 25 wppm. As described herein, the residue of second column 569 may be treated and lower concentrations of ethanol allow the residue to be treated without generating further impurities.

In FIG. 5, the first residue in line 564 is introduced to second column 567 preferably in the top part of column 567, e.g., top half or top third. Feeding first residue in line 564 in a lower portion of second column 567 may unnecessarily increase the energy requirements. Acid column 567 may be a tray column or packed column. In FIG. 5, second column 567 may be a tray column having from 10 to 110 theoretical trays, e.g. from 15 to 95 theoretical trays or from 20 to 75 theoretical trays. Additional trays may be used if necessary to further reduce the ethanol concentration in the residue. In one embodiment, the reboiler duty and column size may be reduced by increasing the number of trays.

Although the temperature and pressure of second column 567 may vary, when at atmospheric pressure the temperature of the second residue in line 569 preferably is from 95° C. to 160° C., e.g., from 100° C. to 150° C. or from 110° C. to 145° C. In one embodiment, first residue in line 564 is preheated to a temperature that is within 20° C. of the temperature of second residue in line 569, e.g., within 15° C. or within 10° C. The temperature of the second distillate exiting in line 571 from second column 567 preferably is from 50° C. to 120° C., e.g., from 75° C. to 118° C. or from 80° C. to 115° C. The temperature gradient may be sharper in the base of second column 569.

The pressure of second column 567 may range from 0.1 kPa to 510 kPa, e.g., from 1 kPa to 475 kPa or from 1 kPa to 375 kPa. In one embodiment, second column 567 operates above atmospheric pressure, e.g., above 170 kPa or above 375 kPa. Second column 567 may be constructed of a material such as 316L SS, Allot 2205 or Hastelloy C, depending on the operating pressure. The reboiler duty and column size for second column 567 remain relatively constant until the ethanol concentration in the second distillate in line 571 is greater than 90 wt. %.

Second column 567 also forms an overhead, which is withdrawn, and which may be condensed and refluxed, for example, at a ratio from 12:1 to 1:12, e.g., from 10:1 to 1:10 or from 8:1 to 1:8. The overhead preferably comprises 85 to 92 wt. % ethanol, e.g., about 87 to 90 wt. % ethanol, with the remaining balance being water and ethyl acetate. In one embodiment, water may be removed prior to recovering the ethanol product as described above. In one embodiment, the overhead, prior to water removal, may comprise less than 15 wt. % water, e.g., less than 10 wt. % water or less than 8 wt. % water. Overhead vapor may be fed to water separator, which may be an adsorption unit, membrane, molecular sieves, extractive column distillation, or a combination thereof.

Exemplary components for the distillate and residue compositions for second column 567 are provided in Table 12 below. It should be understood that the distillate and residue may also contain other components, not listed in Table 12. For example, in optional embodiments, when ethyl acetate is in the feed to reactor 520, second residue in line 569 exemplified in Table 12 may also comprise high boiling point components.

TABLE 12
SECOND COLUMN (FIG. 5)

| | Conc. (wt. %) | Conc. (wt. %) | Conc. (wt. %) |
|---|---|---|---|
| Second Distillate | | | |
| Ethanol | 80 to 96 | 85 to 92 | 87 to 90 |
| Ethyl Acetate | <30 | 0.001 to 15 | 0.005 to 4 |
| Acetaldehyde | <20 | 0.001 to 15 | 0.005 to 4 |
| Water | <20 | 0.001 to 10 | 0.01 to 8 |
| Acetal | <2 | 0.001 to 1 | 0.005 to 0.5 |
| Second Residue | | | |
| Acetic Acid | 0.1 to 55 | 0.2 to 40 | 0.5 to 35 |
| Water | 45 to 99.9 | 55 to 99.8 | 65 to 99.5 |
| Ethyl Acetate | <0.1 | 0.0001 to 0.05 | 0.0001 to 0.01 |
| Ethanol | <5 | 0.002 to 1 | 0.005 to 0.5 |

The weight ratio of ethanol in second distillate in line 571 to ethanol in the second residue in line 569 preferably is at least 35:1. Preferably, second distillate in line 571 is substantially free of acetic acid and may contain, if any, trace amounts of acetic acid.

In one embodiment, ethyl acetate fed to second column 567 may concentrate in the second distillate in line 571. Thus, preferably no ethyl acetate is withdrawn in the second residue in line 569. Advantageously this allows most of the ethyl acetate to be subsequently recovered without having to further process the second residue in line 569.

In one embodiment, as shown in FIG. 5, due to the presence of ethyl acetate in second distillate in line 571, an additional third column 573 may be used. Third column 573, referred to as a “product” column, is used for removing ethyl acetate from second distillate in line 571 and producing an ethanol product in the third residue in line 575. Product column 573 may be a tray column or packed column. In FIG. 5, third column 573 may be a tray column having from 5 to 90 theoretical trays, e.g. from 10 to 60 theoretical trays or from 15 to 50 theoretical trays.

The feed location of second distillate in line 571 may vary depending on ethyl acetate concentration and it is preferred to feed second distillate in line 571 to the upper portion of third column 573. Higher concentrations of ethyl acetate may be fed at a higher location in third column 573. The feed location should avoid the very top trays, near the reflux, to avoid excess reboiler duty requirements for the column and an increase in column size. For example, in a column having 45 actual trays, the feed location should between 10 to 15 trays from the top. Feeding at a point above this may increase the reboiler duty and size of third column 573.

Second distillate in line 571 may be fed to third column 573 at a temperature of up to 70° C., e.g., up to 50° C., or up to 40° C. In some embodiments it is not necessary to further preheat second distillate in line 571.

Ethyl acetate may be concentrated in the third distillate in line 577. Due to the relatively lower amounts of ethyl acetate fed to third column 573, third distillate in line 577 also comprises substantial amounts of ethanol. To recover the ethanol, third distillate in line 577 may be fed to first column 524 as an optional ethyl acetate recycle stream 577. Depending on the ethyl acetate concentration of optional ethyl acetate recycle stream 577 this stream may be introduced above or near the feed point of the liquid stream 562. Depending on the targeted ethyl acetate concentration in the distillate of first column 524 the feed point of optional ethyl acetate recycle stream 577 will vary. Liquid stream 562 and optional ethyl acetate recycle stream 571 collectively comprise the organic feed to first column 524. In one embodiment, organic feed comprises from 1 to 25% of optional ethyl acetate recycle stream 577, e.g., from 3% to 20% or from 5% to 15%. This amount may vary depending on the production of reactor 520 and amount of ethyl acetate to be recycled.

Because ethyl acetate recycle stream 577 increases the demands on the first and second columns, it is preferred that the ethanol concentration in third distillate in line 577 be from 70 to 90 wt. %, e.g., from 72 to 88 wt. %, or from 75 to 85 wt. %. In other embodiments, a portion of third distillate in line 577 may be purged from the system as additional products, such as an ethyl acetate solvent. In addition, ethanol may be recovered from a portion of the third distillate in line 577 using an extractant, such as benzene, propylene glycol, and cyclohexane, so that the raffinate comprises less ethanol to recycle.

The third residue in line 575 from third column 573 may comprise ethanol and optionally any remaining water. In an optional embodiment, the third residue may be further processed to recover ethanol with a desired amount of water, for example, using a further distillation column, adsorption unit, membrane or combination thereof, may be used to further remove water from third residue in line 575, as necessary.

Third column 573 is preferably a tray column as described above and preferably operates at atmospheric pressure. The temperature of the third residue exiting from third column 573 preferably is from 65° C. to 110° C., e.g., from 70° C. to 100° C. or from 75° C. to 80° C. The temperature of the third distillate exiting from third column in line 577 preferably is from 30° C. to 70° C., e.g., from 40° C. to 65° C. or from 50° C. to 65° C.

The pressure of third column 573 may range from 0.1 kPa to 510 kPa, e.g., from 1 kPa to 475 kPa or from 1 kPa to 375 kPa. In some embodiments, third column 573 may operate under a vacuum of less than 70 kPa, e.g., less than 50 kPa, or less than 20 kPa. Decreases in operating pressure substantially decreases column diameter and reboiler duty for third column 176.

Exemplary components for ethanol mixture stream and residue compositions for third column 573 are provided in Table 13 below. It should be understood that the distillate and residue may also contain other components, not listed in Table 13.

TABLE 13
PRODUCT COLUMN (FIG. 3)

| | Conc. (wt. %) | Conc. (wt. %) | Conc. (wt. %) |
|---|---|---|---|
| Third Distillate | | | |
| Ethanol | 70 to 99 | 72 to 95 | 75 to 90 |
| Ethyl Acetate | 1 to 30 | 1 to 25 | 1 to 15 |
| Acetaldehyde | <15 | 0.001 to 10 | 0.1 to 5 |
| Water | <10 | 0.001 to 2 | 0.01 to 1 |
| Acetal | <2 | 0.001 to 1 | 0.01 to 0.5 |
| Third Residue | | | |
| Ethanol | 80 to 99.5 | 85 to 97 | 90 to 95 |
| Water | <3 | 0.001 to 2 | 0.01 to 1 |
| Ethyl Acetate | <1.5 | 0.0001 to 1 | 0.001 to 0.5 |
| Acetic Acid | <0.5 | <0.01 | 0.0001 to 0.01 |

Some of the residues withdrawn from the separation zone(s) comprise acetic acid and water. Depending on the amount of water and acetic acid in some residues of the columns of the FIGS., the residue(s) may be treated in one or more of the following processes. The following are exemplary processes for further treating the residue and it should be understood that any of the following may be used regardless of acetic acid concentration. When the residue comprises a majority of acetic acid, e.g., greater than 70 wt. %, the residue may be recycled to the reactor without any separation of the water. In one embodiment, the residue may be separated into an acetic acid stream and a water stream when the residue comprises a majority of acetic acid, e.g., greater than 50 wt. %. Acetic acid may also be recovered in some embodiments from the residue having a lower acetic acid concentration. The residue may be separated into the acetic acid and water streams by a distillation column or one or more membranes. If a membrane or an array of membranes is employed to separate the acetic acid from the water, the membrane or array of membranes may be selected from any suitable acid resistant membrane that is capable of removing a permeate water stream. The resulting acetic acid stream optionally is returned to the reactor 108. The resulting water stream may be used as an extractive agent or to hydrolyze an ester-containing stream in a hydrolysis unit.

In other embodiments, for example, where the residue comprises less than 50 wt. % acetic acid, possible options include one or more of: (i) returning a portion of the residue to reactor 108, (ii) neutralizing the acetic acid, (iii) reacting the acetic acid with an alcohol, or (iv) disposing of the residue in a waste water treatment facility. It also may be possible to separate a residue comprising less than 50 wt. % acetic acid using a weak acid recovery distillation column to which a solvent (optionally acting as an azeotroping agent) may be added. Exemplary solvents that may be suitable for this purpose include ethyl acetate, propyl acetate, isopropyl acetate, butyl acetate, vinyl acetate, diisopropyl ether, carbon disulfide, tetrahydrofuran, isopropanol, ethanol, and C₃-C₁₂ alkanes. When neutralizing the acetic acid, it is preferred that the residue comprises less than 10 wt. % acetic acid. Acetic acid may be neutralized with any suitable alkali or alkaline earth metal base, such as sodium hydroxide or potassium hydroxide. When reacting acetic acid with an alcohol, it is preferred that the residue comprises less than 50 wt. % acetic acid. The alcohol may be any suitable alcohol, such as methanol, ethanol, propanol, butanol, or mixtures thereof. The reaction forms an ester that may be integrated with other systems, such as carbonylation production or an ester production process. Preferably, the alcohol comprises ethanol and the resulting ester comprises ethyl acetate. Optionally, the resulting ester may be fed to the hydrogenation reactor.

In some embodiments, when the residue comprises very minor amounts of acetic acid, e.g., less than 5 wt. %, the residue may be disposed of to a waste water treatment facility without further processing. The organic content, e.g., acetic acid content, of the residue beneficially may be suitable to feed microorganisms used in a waste water treatment facility.

### Ethanol Composition

The ethanol product may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 14.

TABLE 14
FINISHED ETHANOL COMPOSITIONS

| Component | Conc. (wt. %) | Conc. (wt. %) | Conc. (wt. %) |
|---|---|---|---|
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit such as an adsorption unit, membrane, molecular sieve, or extractive distillation column. In such embodiments, the ethanol concentration of the ethanol product may be higher than indicated in Table 7, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst, such as zeolite catalysts or phosphotungstic acid catalysts, can be employed to dehydrate ethanol, as described in U.S. Pub. Nos. 2010/0030002 and 2010/0030001 and WO2010146332, the entire contents and disclosures of which are hereby incorporated by reference.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, comprising the steps of:
(a) contacting in a reactor under conditions effective to produce a crude acetic acid product:
(i) a carbon monoxide feed; and
(ii) an impure methanol feed comprising more than 0.15 wt. % of impurities;
(b) separating the crude acetic acid product to form an intermediate acetic acid product comprising acetic acid; and
(c) hydrogenating at least a portion of the intermediate acetic acid product over a catalyst and under conditions effective to produce a crude ethanol product.

2. The process of claim 1, wherein the impure methanol feed comprises impurities selected from the group consisting of organic impurities, chlorine-containing compounds, sulfur-containing compounds, and nitrogen-containing compounds.

3. The process of claim 1, wherein the impure methanol feed comprises impurities that are hydrocarbons having more than two carbon atoms and not being selected from the group consisting of methyl acetate, dimethyl ether, or methyl formate.

4. The process of claim 1, wherein the intermediate acetic acid product further comprises at least one of the impurities from the impure methanol feed.

5. The process of claim 1, wherein the intermediate acetic acid product further comprises by-products formed in step (a).

6. The process of claim 5, wherein the by-products comprise by-products derived from the impurities.

7. The process of claim 1, wherein the intermediate acetic acid product further comprises water in an amount from 0.15 wt. % to 25 wt. %.

8. The process of claim 1, wherein the intermediate acetic acid product further comprises water in an amount less than 1500 wppm.

9. The process of claim 1, wherein the intermediate acetic acid product comprises from 0.01 to 10 wt. % methyl acetate.

10. The process of claim 1, wherein the crude ethanol product comprises from 5 wt. % to 72 wt. % ethanol and from 5 wt. % to 40 wt. % water.

11. The process of claim 1, wherein the carbon monoxide feed comprises from 10 wt % to 99.99 wt % carbon monoxide.

12. The process of claim 1, wherein the carbon monoxide feed comprises from 10 wt % to 99.5 wt % carbon monoxide.

13. The process of claim 1, further comprises the step of purifying the intermediate acetic acid product to yield a purified acetic acid product.

14. The process of claim 13, wherein step (c) is performed in a reaction zone and wherein the purified acetic acid product is fed directly to the reaction zone without removing substantially any water therefrom.

15. The process of claim 1, wherein at least one of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

16. The process of claim 1, wherein the intermediate acetic acid product is a sidedraw from a light ends column of a carbonylation process.

17. The process of claim 1, further comprising the steps of:
separating in a first column at least a portion of the crude ethanol product into a first distillate comprising ethanol, water and ethyl acetate, and a first residue comprising acetic acid;
separating in a second column at least a portion of the first distillate into a second distillate comprising ethyl acetate and a second residue comprising ethanol and water;
separating in a third column at least a portion of the second residue into a third distillate comprising ethanol and residual water and a third residue comprising separated water; and
dehydrating at least a portion of the third distillate to form the anhydrous ethanol composition comprising, as formed, less than 1 wt. % water, based on the total weight of the anhydrous ethanol composition.

18. The process of claim 1, further comprising the steps of:
separating at least a portion of the crude ethanol product in a first distillation column to yield a first residue comprising acetic acid and a first distillate comprising ethanol, ethyl acetate, and water;
removing water from at least a portion of the first distillate to yield an ethanol mixture stream comprising less than 10 wt. % water; and
separating a portion of the ethanol mixture stream in a second distillation column to yield a second residue comprising ethanol and a second distillate comprising ethyl acetate.

19. The process of claim 1, further comprising the steps of:
separating a portion of the crude ethanol product in a first distillation column to yield a first distillate comprising ethyl acetate and acetaldehyde, and a first residue comprising ethanol, ethyl acetate, acetic acid and water;
separating a portion of the first residue in a second distillation column to yield a second residue comprising acetic acid and water and a second distillate comprising ethanol and ethyl acetate; and
separating a portion of the second distillate in a third distillation column to yield a third residue comprising ethanol and a third distillate comprising ethyl acetate.
